(19) United States
(12) Patent Application Publication
(10) Pub. No.: US 2021/0379040 A1
(43) Pub. Date: Dec. 9, 2021

(54) COMBINATION TREATMENT OF LIVER DISORDERS

(71) Applicant: Terns Pharmaceuticals, Inc., Foster City, CA (US)

(72) Inventors: Martijn FENAUX, San Mateo, CA (US); Kevin KLUCHER, Bothell, WA (US); Christopher T. JONES, Foster City, CA (US)

(21) Appl. No.: 17/318,997

(22) Filed: May 12, 2021

Related U.S. Application Data
(60) Provisional application No. 63/024,359, filed on May 13, 2020.

(51) Int. Cl. A61K 31/4439; A61K 31/4748; A61K 31/575; A61P 1/16; A61K 31/166; A61K 31/506

(52) U.S. Cl. CPC A61K 31/4439; A61K 31/4748; A61K 31/506; A61P 1/16; A61K 31/166; A61K 31/575

(57) ABSTRACT

Provided herein are methods for treating liver disorders, including non-alcoholic steatohepatitis, and symptoms and manifestations thereof, in a patient which utilize, among others, a combination treatment of an FXR agonist and an SSAO inhibitor.

### CROSS REFERENCE TO RELATED APPLICATIONS

This application claims priority benefit of U.S. Provisional Application No. 63/024,359, filed May 13, 2020, which is incorporated herein by reference in its entirety.

### FIELD OF THE INVENTION

This invention relates to methods and compositions for treating liver disorder in a patient.

### BACKGROUND

Fatty liver disease (FLD) encompasses a spectrum of disease states characterized by excessive accumulation of fat in the liver often accompanied with inflammation. FLD can lead to non-alcoholic fatty liver disease (NAFLD), which may be characterized by insulin resistance. If untreated, NAFLD can progress to a persistent inflammatory response or non-alcoholic steatohepatitis (NASH), progressive liver fibrosis, and eventually to cirrhosis. In Europe and the US, NAFLD is the second most common reason for liver transplantation. Accordingly, the need for treatment is urgent, but due to the lack of obvious symptoms to the patient, patients may lack the motivation to maintain treatment regimens, particularly burdensome treatment regimens, such as injected medicines, medications that are administered many times a day, or any that produce dangerous or irritating side effects. There is currently no approved treatment of NASH.

### BRIEF SUMMARY

Provided herein are methods and compositions for treating a liver disorder in a patient in need thereof. The methods comprise administering to the patient a Farnesoid X Receptor (FXR) agonist and a Semicarbazide-Sensitive Amine Oxidase (SSAO) inhibitor.

In one aspect, the disclosure provides methods of reducing hepatic inflammation in a patient in need thereof, comprising administering to the patient a therapeutically effective amount of a FXR agonist and a therapeutically effective amount of a SSAO inhibitor. The administration of a combination of a FXR agonist and a SSAO inhibitor reduces hepatic inflammation in a patient in need thereof to a significantly greater extent than administration of either agonist by itself. The reduction of hepatic inflammation is characterized by a reduction of leukocyte activation in the liver.

In another aspect, the disclosure provides methods of treating a disease or condition characterized by fibrosis of the liver, comprising administering to the patient a therapeutically effective amount of a FXR agonist and a therapeutically effective amount of a SSAO inhibitor. The administration of a combination of a FXR agonist and a SSAO inhibitor reduces fibrosis in a patient in need thereof to a significantly greater extent than administration of either agonist alone. The reduction of fibrosis is characterized by histological improvement and reduced expression of pro-fibrotic genes in the liver.

In another aspect, the disclosure provides methods of treating a disease or condition characterized by hepatic steatosis, comprising administering to the patient a therapeutically effective amount of a FXR agonist and a therapeutically effective amount of a SSAO inhibitor. It has been discovered that the combination of a FXR agonist and a SSAO inhibitor reduces hepatic steatosis, in part, by regulating genes involved with lipid metabolism and fatty acid transportation. Surprisingly, the FXR agonist potentiates the effect of the SSAO inhibitor in regulating genes associated with lipid metabolism and fatty acid transportation, hence resulting in the reduction of fat (e.g., triglyceride) accumulation in the liver. Accordingly, the administration of a combination of a FXR agonist and a SSAO inhibitor reduces hepatic steatosis in a patient in need thereof to a significantly greater extent than administration of either agent alone.

As set forth herein, the synergy observed when administering the combination of a FXR agonist and a SSAO inhibitor to patients in need thereof allows for the reduction of the dose of either or both the FXR agonist and the SSAO inhibitor relative to when either agonist is administered as a monotherapy. The lower doses of the FXR agonist and the SSAO inhibitor results in an improved therapeutic index and alleviates side effects that are sometimes accompanied with FXR agonism or SSAO inhibition.

In some embodiments, the administration of the FXR agonist and the SSAO inhibitor does not result in pruritus in the patient at a severity of Grade 2 or more. In some embodiments, the administration of the FXR agonist and the SSAO inhibitor does not result in pruritus of Grade 1 or more. In some embodiments, the administration of the FXR agonist and the SSAO inhibitor does not result in pruritus.

In another aspect, the disclosure provide methods of treating or preventing NASH in a patient in need thereof, said method comprising administering to the patient a therapeutically effective amount of a FXR agonist and a therapeutically effective amount of a SSAO inhibitor. In one embodiment, the patient in need thereof is a patient that suffers from fatty liver disease such as NAFLD. In another embodiment, the patient in need thereof is a patient that suffers from insulin resistance syndrome.

In some embodiments, the FXR agonist and the SSAO inhibitor are administered simultaneously. In some such embodiments, the FXR agonist and the SSAO inhibitor are provided as a fixed-dose composition in a single pharmaceutical composition as set forth herein. In other embodiments, the FXR agonist and the SSAO inhibitor are administered sequentially. In some embodiments, either or both of the FXR agonist and the SSAO inhibitor are administered orally.

In some embodiments, the patient has a liver disorder and diabetes mellitus. In some embodiments, the patient has a liver disorder and a cardiovascular disorder. In some embodiments, the treatment period is the remaining lifespan of the patient. In some embodiments, the method does not comprise administering an antihistamine, an immunosuppressant, a steroid, rifampicin, an opioid antagonist, or a selective serotonin reuptake inhibitor (SSRI).

In some embodiments, the FXR agonist is administered once daily. In some embodiments, the FXR agonist is administered twice daily. In some embodiments, the SSAO inhibitor is administered once daily. In some embodiments, the SSAO inhibitor is administered twice daily. In some embodiments, the administration comprises administering the FXR agonist daily for a treatment period of one or more weeks. In some embodiments, the administration comprises administering the SSAO inhibitor daily for a treatment period of one or more weeks. In some embodiments, the administration comprises administering the FXR agonist daily and the SSAO inhibitor daily for a treatment period of one or more weeks.

A variety of different FXR agonists and SSAO inhibitors can be used to achieve the beneficial effects observed on liver disease as discussed herein. For instance, in some embodiments, the FXR agonist administered to the patient in need thereof is obeticholic acid. In some embodiments, the FXR agonist administered to the patient in need thereof is cilofexor. In some embodiments, the FXR agonist administered to the patient in need thereof is tropifexor. In some embodiments, the FXR agonist administered to the patient in need thereof is EYP001 (Vonafexor, proposed INN). In some embodiments, the FXR agonist administered to the patient in need thereof is MET409 (Metacrine). In some embodiments, the FXR agonist administered to the patient in need thereof is MET642 (Metacrine). In some embodiments, the FXR agonist is EDP-305 (by Enanta). In some embodiments, the FXR agonist is EDP-297 (by Enanta).

In some embodiments, the FXR agonist administered to the patient in need thereof is a compound of formula (I):

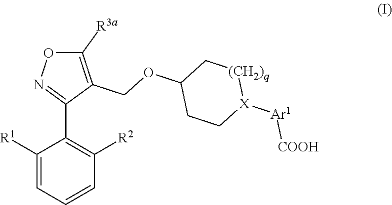

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;
X is CH or N, provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.

In some embodiments, the FXR agonist administered to the patient in need thereof is a compound of formula (I) wherein R¹ is chloro or trifluoromethoxy. In some embodiments, the FXR agonist is a compound of formula (I) wherein R² is hydrogen or chloro. In some embodiments, the FXR agonist is a compound of formula (I) wherein R^{3a} is cyclopropyl or isopropyl. In some embodiments, the FXR agonist is a compound of formula (I) wherein Ar¹ is 5-benzothienyl, 6-benzothienyl, 5-indolyl, 6-indolyl, or 4-phenyl, each of which is optionally substituted with methyl. In some embodiments, the FXR agonist is a compound of formula (I) wherein q is 1 and X is N.

In some embodiments, the FXR agonist is

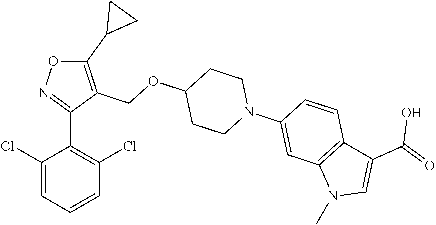 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.

In some embodiments, the SSAO inhibitor administered to the patient in need thereof is a compound of Formula (II)

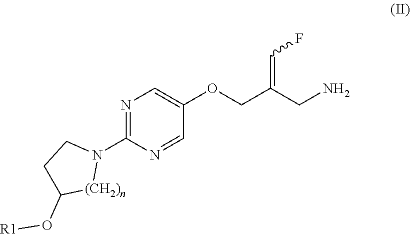

wherein:
n is 1 or 2; and

### R1 is H or —CH₃,

or a pharmaceutically acceptable salt thereof.

In some embodiments, the SSAO inhibitor administered to the patient in need thereof is a compound of Formula (II), where n is 1, or a pharmaceutically acceptable salt thereof. In another embodiment, the SSAO inhibitor is a compound of Formula (II), where n is 2, or a pharmaceutically acceptable salt thereof.

In some embodiments, the SSAO inhibitor administered to the patient in need thereof is a compound of Formula (II), where R1 is H, or a pharmaceutically acceptable salt thereof. In yet another embodiment, the present invention provides a compound of Formula (II), where R1 is —CH₃, or a pharmaceutically acceptable salt thereof.

In some embodiments, the SSAO inhibitor administered to the patient in need thereof is

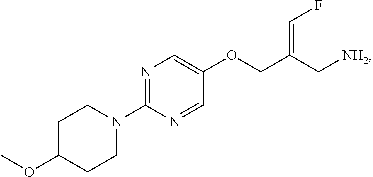 [SMILES: COC1CCN(c2ncc(OC/C(=C/F)CN)cn2)CC1]

or a pharmaceutically acceptable salt thereof.

In some embodiments, provided are methods of treating a liver disorder in a patient in need thereof with a Farnesoid X Receptor (FXR) agonist and a Semicarbazide-Sensitive Amine Oxidase (SSAO) inhibitor, comprising administering a therapeutically effective amount of the FXR agonist, wherein the FXR agonist is

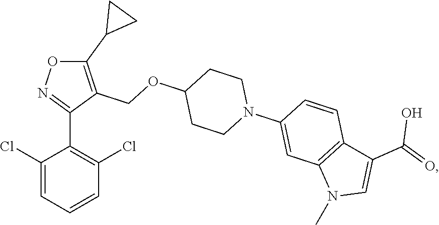 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and administering a therapeutically effective amount of the SSAO inhibitor, wherein the SSAO inhibitor is

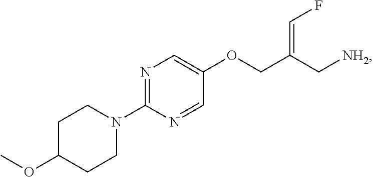 [SMILES: COC1CCN(c2ncc(OC/C(=C/F)CN)cn2)CC1]

or a pharmaceutically acceptable salt thereof, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

### BRIEF DESCRIPTION OF THE DRAWINGS

FIG. 1A shows plasma concentrations of Compound 1 at various time points after intravenous (IV) administration to rats (1 mg/kg), dogs (1 mg/kg) and monkeys (0.3 mg/kg).

FIG. 1B shows plasma concentrations of Compound 1 at various time points after oral administration to mice (10 mg/kg), rats (10 mg/kg), dogs (3 mg/kg) and monkeys (5 mg/kg).

FIG. 2A shows the liver to plasma ratio of the concentration of Compound 1, obeticholic acid (OCA), cilofexor, or tropifexor after 2 mg/kg IV administration to Sprague-Dawley (SD) rats.

FIG. 2B shows the tissue to plasma ratio of the concentration of Compound 1 for kidney, lung, and liver after 2 mg/kg IV administration of Compound 1 to SD rats with or without co-administration of rifampicin.

FIG. 3 shows the tissue distribution of radiolabeled Compound 1 in plasma, liver, small intestine, cecum, kidney, lungs, heart, and skin after 5 mg/kg oral administration of Compound 1 to Long-Evans rats.

FIG. 4 shows the pharmacodynamics of Compound 1 administration, as measured by 7-alpha-hydroxy-4-cholesten-3-one (7AC4), after administration of 0.3 mg/kg, 1 mg/kg or 5 mg/kg oral dose to cynomolgus monkeys.

FIG. 5A shows the pharmacokinetics of Compound 1 administration, after administration of 1 mg/kg oral dose for one day, or 7 consecutive daily doses, to cynomolgus monkeys.

FIG. 5B shows the pharmacodynamics of Compound 1 administration, as measured by 7-alpha-hydroxy-4-cholesten-3-one (7AC4), after administration of 1 mg/kg oral dose for one day, or 7 consecutive daily doses, to cynomolgus monkeys.

FIG. 6 shows RT-qPCR results measuring liver SHP1, liver OSTb, ileum SHP1, and ileum FGF15 RNA expression after administering 10 mg/kg Compound 1, 30 mg/kg OCA, or vehicle control to C57BL/6 mice.

FIG. 7A shows the number of differentially expressed genes (vs. vehicle-treated: fold-change >1.5-fold; p<0.05) modulated by the administration of 10 mg/kg Compound 1 (500 total genes modulated) or 30 mg/kg OCA to C57BL/6 mice (44 total genes modulated), as well as the shared number of differentially expressed genes that are modulated by both compounds (37 total genes).

FIG. 7B shows average expression levels (as shown by CPM value) of select FXR-related genes in C5BL/6 mice treated with 10 mg/kg Compound 1 or 30 mg/kg OCA, or a vehicle control.

FIG. 7C shows the number of pathways enriched (p<0.05) by the administration of 10 mg/kg Compound 1 (32 pathways) or 30 mg/kg OCA to C57BL/6 mice (6 pathways), as well as the number of enriched pathways by either compound (2 pathways).

FIG. 7D shows the 25 pathways most statistically enriched upon administration of 10 mg/kg Compound 1 to C57BL/6 mice, and compares the enrichment of those pathways to the enrichment upon administration of 30 mg/kg OCA.

FIG. 8 shows the design of a study testing the efficacy of Compound 1 on a mouse model of NASH.

FIG. 9 shows the NAFLD Activity Score (NAS) of control mice and mice treated with 10, 30, and 100 mg/kg Compound 1.

FIG. 10A shows the steatosis score of control mice and NASH mice treated with 10, 30, and 100 mg/kg Compound 1.

FIG. 10B shows the inflammation score of control mice and NASH mice treated with 10, 30, and 100 mg/kg Compound 1.

FIG. 10C shows the ballooning score of control mice and NASH mice treated with 10, 30, and 100 mg/kg Compound 1.

FIG. 11A shows a histological section of fibrosis in control mice and NASH mice treated with 100 mg/kg Compound 1.

FIG. 11B shows the amount of fibrosis in control mice and NASH mice treated with 10, 30, and 100 mg/kg Compound 1.

FIG. 12A shows the serum alanine amino transferase (ALT) levels of control mice and NASH mice treated with 10, 30, and 100 mg/kg Compound 1.

FIG. 12B shows aspartate amino transferase (AST) of control mice and NASH mice treated with 10, 30, and 100 mg/kg Compound 1.

FIG. 12C shows serum triglyceride levels of control mice and NASH mice treated with 10, 30, and 100 mg/kg Compound 1.

FIG. 12D shows serum total cholesterol levels of control mice and NASH mice treated with 10, 30, and 100 mg/kg Compound 1.

FIG. 13A shows liver triglyceride levels of control mice and NASH mice treated with 10, 30, and 100 mg/kg Compound 1.

FIG. 13B shows representative histology of steatosis assessment for control mice and NASH mice treated with 100 mg/kg Compound 1.

FIG. 14A shows COL1A1 expression in the liver in control mice and NASH mice treated with 10, 30, and 100 mg/kg Compound 1.

FIG. 14B shows expression levels of inflammatory genes in control mice and NASH mice treated with 30 mg/kg Compound 1.

FIG. 14 C shows expression of fibrosis genes in control mice and NASH mice treated with 30 mg/kg Compound 1.

FIG. 15A shows the plasma SSAO-specific amine oxidase activity compared to baseline of healthy volunteers administered a single dose of placebo or 1, 3, 6, or 10 mg of Compound 2 at 4 hours and 168 hours post dose. FIG. 15B shows a time course of plasma total amine oxidase activity compared to baseline of healthy volunteers administered a single dose of placebo or 1, 3, 6, or 10 mg of Compound 2. FIG. 15C shows a time course of the level of Compound 2 after with a single dose of placebo or 1, 3, 6, or 10 mg in healthy volunteers. FIG. 15D shows a time course of the level of plasma methylamine after a single dose of placebo or 1, 3, 6, or 10 mg of Compound 2 in healthy volunteers.

FIG. 16 shows the levels of Treg and M2 macrophage liver infiltration determined by single-sample gene set enrichment analysis. The analysis was performed on liver RNA sequencing data of CDHFD rats administered NaNO₂ and treated with Compound 1, Compound 2, or the combination of Compound 1 and Compound 2 (*p-value <0.05; ***p-value <0.001).

FIG. 17 shows expression analysis by RNA sequencing for markers of Treg and M2 macrophages in the liver of CDHFD rats administered NaNO₂ and treated with Compound 1, Compound 2, or the combination of Compound 1 and Compound 2. Ikzf2, IKAROS Family Zinc Finger 2 (Treg marker); Foxp3, Forkhead Box P3 (Treg marker); Cd163 (M2 macrophage marker). (*p-value <0.05; **p-value <0.01).

FIG. 18 shows the number and overlap of differentially expressed genes (DEGs) identified by RNA sequencing analysis in the liver of CDHFD rats administered NaNO₂ and treated with Compound 1, Compound 2, or the combination of Compound 1 and Compound 2, relative to a vehicle NASH control using fold-change and p-value cutoffs of ≥1.5 and 0.01, respectively.

### DETAILED DESCRIPTION

### Definitions

As used herein, the following definitions shall apply unless otherwise indicated. Further, if any term or symbol used herein is not defined as set forth below, it shall have its ordinary meaning in the art.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this invention.

“Combination therapy” or “combination treatment” refers to the use of two or more drugs or agents in treatment, e.g., the use of a compound of formula (I) or (II) as utilized herein together with another agent useful to treat liver disorders, such as NAFLD, NASH, and symptoms and manifestations of each thereof is a combination therapy. Administration in “combination” refers to the administration of two agents (e.g., a compound of formula (I) or (II) as utilized herein, and another agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agents can also be formulated in a single pharmaceutically acceptable composition. A non-limiting example of such a single composition is an oral composition or an oral dosage form. For example, and without limitation, it is contemplated that a compound of formula (I) or (II) can be administered in combination therapy with another agent in accordance with the present invention.

The term “excipient” as used herein means an inert or inactive substance that may be used in the production of a drug or pharmaceutical, such as a tablet containing a compound of the invention as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Patient” refers to mammals and includes humans and non-human mammals. Examples of patients include, but are not limited to mice, rats, hamsters, guinea pigs, pigs, rabbits, cats, dogs, goats, sheep, cows, and humans. In some embodiments, patient refers to a human.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Treatment” or “treating” refers to an approach for obtaining beneficial or desired results including clinical results. For purposes of this invention, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of the invention contemplate any one or more of these aspects of treatment.

As used herein, “delaying” development of a disease means to defer, hinder, slow, retard, stabilize and/or postpone development of the disease and/or slowing the progression or altering the underlying disease process and/or course once it has developed. This delay can be of varying lengths of time, depending on the history of the disease and/or individual being treated. As is evident to one skilled in the art, a sufficient or significant delay can, in effect, encompass prevention, in that the individual does not develop clinical symptoms associated with the disease. A method that “delays” development of a disease is a method that reduces probability of disease development in a given time frame and/or reduces extent of the disease in a given time frame, when compared to not using the method, including stabilizing one or more symptoms resulting from the disease.

An individual who is “at risk” of developing a disease may or may not have detectable disease, and may or may not have displayed detectable disease prior to the treatment methods described herein. “At risk” denotes that an individual has one or more so-called risk factors, which are measurable parameters that correlate with development of a disease. An individual having one or more of these risk factors has a higher probability of developing the disease than an individual without these risk factor(s). These risk factors include, but are not limited to, age, sex, race, diet, history of previous disease, presence of precursor disease and genetic (i.e., hereditary) considerations. Compounds may, in some embodiments, be administered to a subject (including a human) who is at risk or has a family history of the disease or condition.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH₂—), ethylene (—CH₂CH₂— or —CH(Me)-), propylene (—CH₂CH₂CH₂— or —CH(Me)CH₂—, or —CH(Et)-) and the likes.

“Alkenyl” refers to straight or branched monovalent hydrocarbyl groups having from 2 to 6 carbon atoms and preferably 2 to 4 carbon atoms and having at least 1 and preferably from 1 to 2 sites of vinyl (>C═C<) unsaturation. Such groups are exemplified, for example, by vinyl, allyl, and but-3-en-1-yl. Included within this term are the cis and trans isomers or mixtures of these isomers. Cₓ alkenyl refers to an alkenyl group having x number of carbon atoms.

“Alkynyl” refers to straight or branched monovalent hydrocarbyl groups having from 2 to 6 carbon atoms and preferably 2 to 3 carbon atoms and having at least 1 and preferably from 1 to 2 sites of acetylenic (—C≡C—) unsaturation. Examples of such alkynyl groups include acetylenyl (—C≡CH), and propargyl (—CH₂C≡CH). Cₓ alkynyl refers to an alkynyl group having x number of carbon atoms.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring. “Substituted cycloalkyl” refers to a cycloalkyl group having from 1 to 5 or preferably 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

### FXR Agonists

Suitable FXR agonists that can be used in accordance with the methods described herein include, but are not limited to obeticholic acid, cilofexor, tropifexor, EYP001 (Vonafexor, proposed INN), MET409 (Metacrine), MET642 (Metachrine), EDP-305 (by Enanta), EDP-297 (by Enanta), and a compound of formula (I) or a pharmaceutically acceptable salt. The compound of formula (I) is disclosed in US 2010/0152166, the content of which is incorporated by reference in its entirety, and specifically with respect to the compound of formula (I) or a pharmaceutically acceptable salt or enantiomer thereof, as well as methods of making and using the foregoing.

In some embodiments, the FXR agonist is a compound of formula (I)

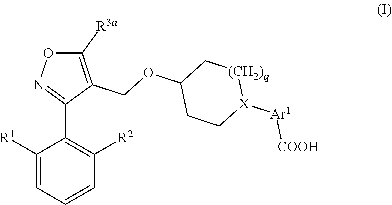

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;

### X is CH or N,

provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl, or a pharmaceutically acceptable salt thereof.

In some embodiments, the FXR agonist is a compound of formula (I), wherein R¹ is chloro or trifluoromethoxy; and R² is hydrogen or chloro.

In some embodiments, the FXR agonist is a compound of formula (I), wherein R^{3a} is cyclopropyl or isopropyl.

In some embodiments, the FXR agonist is a compound of formula (I), wherein Ar¹ is 5-benzothienyl, 6-benzothienyl, 5-indolyl, 6-indolyl, or 4-phenyl, each of which is optionally substituted with methyl.

In some embodiments, the FXR agonist is a compound of formula (I), wherein q is 1; and X is N.

In some embodiments, the FXR agonist is a compound of formula 1:

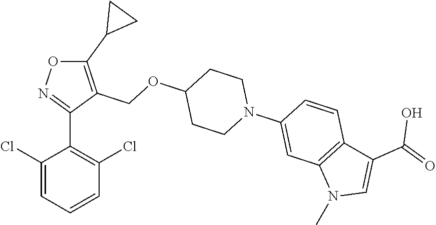 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof. “Compound 1” refers to the compound of formula 1.

### SSAO Inhibitors

Suitable SSAO inhibitors that can be used in accordance with the methods described herein include, but are not limited to PXS-4728A (BI-1467335) and a compound of formula (II) or a pharmaceutically acceptable salt. The compound of formula (II) is disclosed in US 2018/0297987, the content of which is incorporated by reference in its entirety, and specifically with respect to the compound of formula (II) or a pharmaceutically acceptable salt or enantiomer thereof, as well as methods of making and using the foregoing.

In some embodiments, the SSAO inhibitor is a compound of Formula (II)

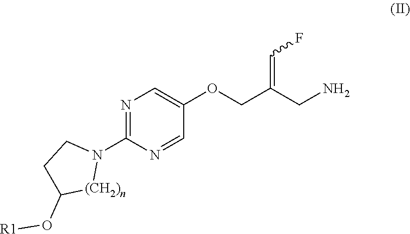

or a pharmaceutically acceptable salt thereof, wherein:
n is 1 or 2; and

### R1 is H or —CH₃.

The bond to fluorine, which is illustrated as [symbol] , indicates that the fluorine atom and the methoxypyrimidine group can be either Z (zusammen, together) or E (entgegen, opposite) relative to each other (Brecher, J., et al., “Graphical Representation of Stereochemical Configuration”, Pure and Appl. Chem, 2006, 78(10) 1897, at 1959). The structure illustrated by Formula (II) includes compounds with the Z stereochemical configuration, the E stereochemical configuration, or a mixture of compounds in the Z or E stereochemical configurations. Preferred compounds of the invention have the E stereochemical configuration.

In one form, the compounds of Formula (II) are presented as a free base. In other form, the compounds of Formula (II) are presented as acid addition salts, such as a mono or di HCl addition salt(s) or a sulfonate salt, preferable a 4-methylbenzenesulfonate (a tosylate salt).

In some embodiments, the SSAO inhibitor is a compound of formula (IIa)

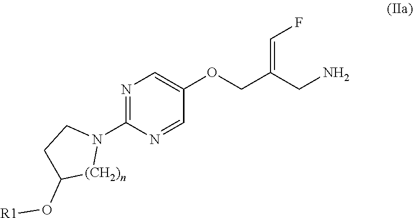

or a pharmaceutically acceptable salt thereof, wherein:
n is 1 or 2; and

### R1 is H or —CH₃.

In some embodiments, the SSAO inhibitor

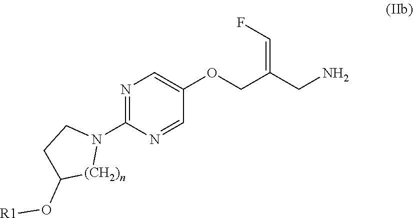

or a pharmaceutically acceptable salt thereof, wherein:
n is 1 or 2; and

### R1 is H or —CH₃.

In some embodiments, the SSAO inhibitor is a compound of formula (II), (IIa) or (IIb) and n is 2.

In some embodiments, the SSAO inhibitor is a compound of formula (II), (IIa) or (IIb) and R1 is CH₃.

In some embodiments, the SSAO inhibitor is a compound of formula 2:

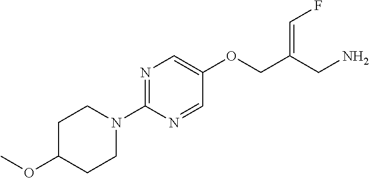 [SMILES: COC1CCN(c2ncc(OC/C(=C/F)CN)cn2)CC1]

a pharmaceutically acceptable salt thereof. “Compound 2” refers to the compound of formula 2.

### Pharmaceutically Acceptable Compositions and Formulations

Pharmaceutically acceptable compositions or simply “pharmaceutical compositions” of any of the compounds detailed herein are embraced by this invention. Thus, the invention includes pharmaceutical compositions comprising an FXR agonist (such as the compound of Formula (I) or a pharmaceutically acceptable salt thereof), an SSAO inhibitor (such as the compounds of Formula (II) or a pharmaceutically acceptable salt thereof), and a pharmaceutically acceptable carrier or excipient. In some embodiments, the pharmaceutically acceptable salt is an acid addition salt, such as a salt formed with an inorganic or organic acid. Pharmaceutical compositions according to the invention may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the invention embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21S ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

Compositions comprising two compounds utilized herein are described. Any of the compounds described herein can be formulated in a tablet in any dosage form described herein.

The present disclosure further encompasses kits (e.g., pharmaceutical packages). The kit provided may comprise the pharmaceutical compositions or the compounds described herein and containers (e.g., drug bottles, ampoules, bottles, syringes and/or subpackages or other suitable containers). In some embodiments, the kit includes a container comprising the FXR agonist (such as the compound of Formula (I) or a pharmaceutically acceptable salt thereof) and the SSAO inhibitor (such as the compound of (II) or a pharmaceutically acceptable salt thereof). In other embodiments, the kit includes a first container comprising FXR agonist (such as the compound of Formula (I) or a pharmaceutically acceptable salt thereof) and a second container comprising the SSAO inhibitor (such as the compound of (II) or a pharmaceutically acceptable salt thereof).

In some embodiments, the composition comprises the FXR agonist and the SSAO inhibitor as described herein. In some embodiments, such a composition includes a compound of formula (I), or a pharmaceutically acceptable salt thereof, and a compound of formula (II), or a pharmaceutically acceptable salt thereof. In some embodiments, provided herein is a dosage form comprises a therapeutically effective amount of a compound of formula (I), or a pharmaceutically acceptable salt thereof, and a therapeutically effective amount of a compound of formula (II), or a pharmaceutically acceptable salt thereof. In some embodiments, the compound of formula (I), or a pharmaceutically acceptable salt thereof, is Compound 1, and the compound of formula (II), or a pharmaceutically acceptable salt thereof, is Compound 2 as described herein.

### Methods of Use and Uses

Compounds and compositions described herein may in some aspects be used in treatment of liver disorders. In some embodiments, the method of treating a liver disorder in a patient in need thereof comprises administering to the patient a Farnesoid X Receptor (FXR) agonist and a Semicarbazide-Sensitive Amine Oxidase (SSAO) inhibitor. In some embodiments, the FXR agonist is a compound of Formula (I), or a pharmaceutically acceptable salt thereof, and the SSAO inhibitor is a compound of Formula (II), or a pharmaceutically acceptable salt thereof. In one embodiment, the compound of Formula (I), or a pharmaceutically acceptable salt thereof, is Compound 1, and the compound of Formula (II), or a pharmaceutically acceptable salt thereof, is Compound 2 as described herein. Without being bound by theory, it is believed that the combination of an FXR agonist and an SSAO inhibitor in accordance with the methods described herein may effectively provide treatment as compared to monotherapies and thus reduce dose-dependent adverse effects that may accompany monotherapy treatment.

Liver disorders include, without limitation, liver inflammation, fibrosis, and steatohepatitis. In some embodiments, the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In certain embodiments, the liver disorder is selected from: liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, the method of treating a liver disorder in a patient in need thereof, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Provided herein are methods of treating a liver disorder in a patient (e.g., a human patient) in need thereof with an FXR agonist and an SSAO inhibitor, comprising administering a therapeutically effective amount of the FXR agonist and a therapeutically effective amount of the SSAO inhibitor, wherein the liver disorder is selected from liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the FXR agonist is a compound of Formula (I) or a pharmaceutically acceptable salt thereof and the SSAO inhibitor is a compound of formula (II) or a pharmaceutically acceptable salt thereof. In some embodiments, the compound of formula (I), or a pharmaceutically acceptable salt thereof, is Compound 1, and the compound of formula (II), or a pharmaceutically acceptable salt thereof, is Compound 2 as described herein.

Also provided herein are methods of impeding or slowing the progression of non-alcoholic fatty liver disease (NAFLD) to non-alcoholic steatohepatitis (NASH) in a patient (e.g., a human patient) in need thereof comprising administering an FXR agonist (such as the compound of Formula (I) or a pharmaceutically acceptable salt thereof) and an SSAO inhibitor (such as the compounds of Formula (II) or a pharmaceutically acceptable salt thereof). In some embodiments, the methods comprises administering a therapeutically effective amount of a compound of formula (I), or a pharmaceutically acceptable salt thereof, and a therapeutically effective amount of a compound of formula (II) or a pharmaceutically acceptable salt thereof. Also provided herein are methods of impeding or slowing the progression of NASH in a patient (e.g., a human patient) in need thereof comprising administering an FXR agonist (such as the compound of Formula (I) or a pharmaceutically acceptable salt thereof) and an SSAO inhibitor (such as the compounds of Formula (II) or a pharmaceutically acceptable salt thereof). In some embodiments, the methods comprises administering a therapeutically effective amount of a compound of formula (I), or a pharmaceutically acceptable salt thereof, and a therapeutically effective amount of a compound of formula (II) or a pharmaceutically acceptable salt thereof.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of the compound of formula (I), in particular the preference for liver over skin tissue is a striking and unpredicted observation that makes it more likely that the compound will not cause pruritus in the skin, a theory that has been substantiated by human trials thus far.

Accordingly, provided herein are methods of treating a liver disorder in a patient in need thereof (e.g., a human patient) with an FXR agonist and an SSAO inhibitor, wherein the FXR is a compound of Formula (I), or a pharmaceutically acceptable salt thereof, which preferentially distributes in liver tissue over one or more of kidney, lung, heart, and skin.

In some embodiments, the administration results in a liver concentration to plasma concentration ratio of the compound of Formula (I) of 10 or greater, such as 11 or greater, 12 or greater, 13 or greater, 14 or greater, or 15 or greater.

In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.” Accordingly, provided herein are methods of treating a liver disorder in a patient (e.g., a human patient) in need thereof with an FXR agonist that does not result in detectable pruritus in the patient in need thereof.

In some embodiments, provided herein are methods of treating a liver disorder in a patient in need thereof with an FXR agonist (such as the compound of Formula (I) or a pharmaceutically acceptable salt thereof) and an SSAO inhibitor (such as the compounds of Formula (II) or a pharmaceutically acceptable salt thereof), wherein the FXR agonist does not activate TGR5 signaling. In some embodiments, the level of an FXR-regulated gene is increased. In some embodiments, the level of small heterodimer partner (SHP), bile salt export pump (BSEP) and fibroblast growth factor 19 (FGF19) is increased.

In some embodiments, provided herein a method of reducing liver damage comprising administering an FXR agonist (such as the compound of Formula (I) or a pharmaceutically acceptable salt thereof) and an SSAO inhibitor (such as the compounds of Formula (II) or a pharmaceutically acceptable salt thereof), to an individual in need thereof, wherein fibrosis is reduced. In some embodiments, the level of expression of one or more markers for fibrosis is reduced. In some embodiments, the level of Ccr2, Co11a1, Co11a2, Co11a3, Cxcr3, Dcn, Hgf, I11a, Inhbe, Lox, Lox11, Lox12, Lox13, Mmp2, pdgfb, Plau, Serpine1, Perpinh1, Snai, Tgfb1, Tgfb3, Thbs1, Thbs2, Timp2, and/or Timp3 expression is reduced. In some embodiments the level of collagen is reduced. In some embodiments, the level of collagen fragments is reduced. In some embodiments, the level of expression of the fibrosis marker is reduced at least 2, at least 3, at least 4, or at least 5-fold. In some embodiments, the level of expression of the fibrosis marker is reduced about 2-fold, about 3-fold, about 4-fold, or about 5-fold.

In some embodiments, provided herein a method of reducing liver damage comprising administering an FXR agonist (such as the compound of Formula (I) or a pharmaceutically acceptable salt thereof) and an SSAO inhibitor (such as the compounds of Formula (II) or a pharmaceutically acceptable salt thereof), to an individual in need thereof, wherein inflammation is reduced. In some embodiments, one or more markers of inflammation are reduced. In some embodiments, the level of expression of Adgre1, Ccr2, Ccr5, I11A, and/or Tlr4 is reduced. In some embodiments, the level of expression of the inflammation marker is reduced at least 2-, at least 3-, at least 4-, or at least 5-fold. In some embodiments, the level of expression of the inflammation marker is reduced about 2-fold, about 3-fold, about 4-fold, or about 5-fold.

In a patient, alkaline phosphatase, gamma-glutamyl transferase (GGT), alanine aminotransferase (ALT) and/or aspartate aminotransferase (AST) levels can be elevated. In some embodiments, provided herein a method of reducing liver damage comprising administering an FXR agonist (such as the compound of Formula (I) or a pharmaceutically acceptable salt thereof) and an SSAO inhibitor (such as the compounds of Formula (II) or a pharmaceutically acceptable salt thereof), wherein the GGT, ALT, and/or AST levels are elevated prior to treatment with the FXR agonist. In some embodiments, the FXR agonist is a compound of Formula (I) or a pharmaceutically acceptable salt thereof. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a compound of Formula (I), or a pharmaceutically acceptable salt thereof, reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, provided herein are methods of treating a liver disorder in a patient in need thereof with an FXR agonist (such as the compound of Formula (I) or a pharmaceutically acceptable salt thereof) and an SSAO inhibitor (such as the compound of Formula (II) or a pharmaceutically acceptable salt thereof), wherein the SSAO inhibitor selectively inhibits SSAO. In some embodiments, the SSAO inhibitor is a compound of Formula (II) or a pharmaceutically acceptable salt thereof. Accordingly, in some embodiments, MAO-A (Monoamine oxidase A) is not inhibited. In some embodiments, MAO-B (Monoamine oxidase B) is not inhibited. In some embodiments MAO-A and MAO-B are not inhibited.

In some embodiments, the IC₅₀ for a compound of Formula (II), or a pharmaceutically acceptable salt thereof, is at least 100-fold lower for SSAO than for MAO-A and/or MAO-B. In some embodiments, the IC₅₀ for the compound is at least 1,000-fold lower for SSAO than for MAO-A and/or MAO-B. In some embodiments, the IC₅₀ for the compound is at least 10,000-fold lower for SSAO than for MAO-A and/or MAO-B. In some embodiments, the IC₅₀ for the compound is between 100 to 10,000-fold lower for SSAO than for MAO-A and/or MAO-B. In some embodiments, the IC₅₀ for the compound is between 100 to 1,000-fold lower for SSAO than for MAO-A or MAO-B. In some embodiments, the IC₅₀ for the compound is at least 100-fold or at least 1,000-fold or at least 10,000-fold or between 100 to 10,000-fold or between 100 to 1,000-fold lower for SSAO than for MAO-A and for MAO-B.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, treatment in accordance with the methods provided herein results in a reduced NAFLD Activity (NAS) score in a patient. For example, in some embodiments, steatosis, inflammation, and/or ballooning is reduced upon treatment. In some embodiments, the methods of treatment provided herein reduce liver fibrosis. In some embodiments, the methods reduce serum triglycerides. In some embodiments, the methods reduce liver triglycerides.

In some embodiments, the patient is at risk of developing an adverse effect prior to the administration in accordance with the methods provided herein. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient suffered from pruritus during at least one of the one or more prior therapies.

In some embodiments, the methods described herein do not comprise treating pruritus in the patient. In some embodiments, the methods do not comprise administering an antihistamine, an immunosuppressant, a steroid (such as a corticosteroid), rifampicin, an opioid antagonist, or a selective serotonin reuptake inhibitor (SSRI).

In some embodiments, the therapeutically effective amounts of either the FXR agonist or the SSAO inhibitor, or both are below the level that induces an adverse effect in the patient, such as below the level that induces pruritus, such as grade 2 or grade 3 pruritus.

In some embodiments, the FXR agonist and the SSAO inhibitor are administered simultaneously. In some such embodiments, the FXR agonist and the SSAO inhibitor can be provided in a single pharmaceutical composition. In other embodiments, the FXR agonist and the SSAO inhibitor are administered sequentially.

Also provided herein are dosing regimens for administering an FXR agonist (such as the compound of Formula (I) or a pharmaceutically acceptable salt thereof) and an SSAO inhibitor (such as the compounds of Formula (II) or a pharmaceutically acceptable salt thereof), to an individual in need thereof. In some embodiments, the therapeutically effective amounts of the FXR agonist (such as the compound of Formula (I) or a pharmaceutically acceptable salt thereof) and the SSAO inhibitor (such as the compounds of Formula (II) or a pharmaceutically acceptable salt thereof) are independently 500 μg/day-600 mg/day. In some embodiments, the therapeutically effective amounts are independently 500 μg/day-300 mg/day. In some embodiments, the therapeutically effective amounts are independently 500 μg/day-150 mg/day. In some embodiments, the therapeutically effective amounts are independently 500 μg/day-100 mg/day. In some embodiments, the therapeutically effective amounts are independently 500 μg/day-20 mg/day. In some embodiments, the therapeutically effective amounts are independently 1 mg/day-600 mg/day. In some embodiments, the therapeutically effective amounts are independently 1 mg/day-300 mg/day. In some embodiments, the therapeutically effective amounts are independently 1 mg/day-150 mg/day. In some embodiments, the therapeutically effective amounts are independently 1 mg/day-100 mg/day. In some embodiments, the therapeutically effective amounts are independently 1 mg/day-20 mg/day. In some embodiments, the therapeutically effective amounts are independently 5 mg/day-300 mg/day. In some embodiments, the therapeutically effective amounts are independently 5 mg/day-150 mg/day. In some embodiments, the therapeutically effective amounts are independently 5 mg/day-100 mg/day. In some embodiments, the therapeutically effective amounts are independently 5 mg/day-20 mg/day. In some embodiments, the therapeutically effective amounts are independently 5 mg/day-15 mg/day. In some embodiments, the therapeutically effective amounts are independently 10 mg/day-300 mg/day. In some embodiments, the therapeutically effective amounts are independently 10 mg/day-150 mg/day. In some embodiments, the therapeutically effective amounts are independently 10 mg/day-100 mg/day. In some embodiments, the therapeutically effective amounts are independently 10 mg/day-30 mg/day. In some embodiments, the therapeutically effective amounts are independently 10 mg/day-20 mg/day. In some embodiments, the therapeutically effective amounts are independently 10 mg/day-15 mg/day. In some embodiments, the therapeutically effective amounts are independently 25 mg/day-300 mg/day. In some embodiments, the therapeutically effective amounts are independently 25 mg/day-150 mg/day. In some embodiments, the therapeutically effective amounts are independently 25 mg/day-100 mg/day. In some embodiments, the therapeutically effective amounts are independently 500 μg/day-5 mg/day. In some embodiments, the therapeutically effective amounts are independently 500 μg/day-4 mg/day. In some embodiments, the therapeutically effective amounts are independently 5 mg/day-600 mg/day. In another embodiment, the therapeutically effective amounts are independently 75 mg/day-600 mg/day. In one embodiment, the compound of Formula (I), or a pharmaceutically acceptable salt thereof, is Compound 1, and the compound of Formula (II), or a pharmaceutically acceptable salt thereof, is Compound 2 as described herein.

The dosage amount of a compound as described herein is determined based on the free base of a compound. In some embodiments, about 1 mg to about 30 mg of the FXR agonist (such as the compound of Formula (I) or a pharmaceutically acceptable salt thereof) is administered to the individual. In some embodiments, about 1 mg to about 5 mg of the compound is administered to the individual. In some embodiments about 1 mg to about 3 mg of the compound is administered to the individual. In some embodiments about 5 mg to about 10 mg of the compound is administered to the individual. In some embodiments, about 10 mg to about 15 mg of the compound is administered to the individual. In some embodiments, about 15 mg to about 20 mg of the compound is administered to the individual. In some embodiments, about 20 mg to about 25 mg of the compound is administered to the individual. In some embodiments, about 25 mg to about 30 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, about 15 mg of the compound is administered to the individual. In some embodiments, about 20 mg of the compound is administered to the individual. In some embodiments, about 25 mg of the compound is administered to the individual. In some embodiments, about 30 mg of the compound is administered to the individual. In one embodiment, the compound is Compound 1 as described herein.

In some embodiments, about 1 mg to about 30 mg of the SSAO inhibitor (such as the compound of Formula (II) or a pharmaceutically acceptable salt thereof) is administered to the individual. In some embodiments, about 1 mg to about 5 mg of the compound is administered to the individual. In some embodiments about 1 mg to about 3 mg of the compound is administered to the individual. In some embodiments about 5 mg to about 10 mg of the compound is administered to the individual. In some embodiments, about 10 mg to about 15 mg of the compound is administered to the individual. In some embodiments, about 15 mg to about 20 mg of the compound is administered to the individual. In some embodiments, about 20 mg to about 25 mg of the compound is administered to the individual. In some embodiments, about 25 mg to about 30 mg of the compound is administered to the individual. In some embodiments, about 1 mg of the compound is administered to the individual. In some embodiments, about 2 mg of the compound is administered to the individual. In some embodiments, about 3 mg of the compound is administered to the individual. In some embodiments, about 4 mg of the compound is administered to the individual. In some embodiments, about 5 mg of the compound is administered to the individual. In some embodiments, about 6 mg of the compound is administered to the individual. In some embodiments, about 7 mg of the compound is administered to the individual. In some embodiments, about 8 mg of the compound is administered to the individual. In some embodiments, about 9 mg of the compound is administered to the individual. In some embodiments, about 10 mg of the compound is administered to the individual. In some embodiments, about 15 mg of the compound is administered to the individual. In some embodiments, about 20 mg of the compound is administered to the individual. In some embodiments, about 25 mg of the compound is administered to the individual. In some embodiments, about 30 mg of the compound is administered to the individual. In one embodiment, the compound is Compound 2 as described herein.

The treatment period generally can be one or more weeks. In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

The administration of the FXR agonist (such as the compound of Formula (I) or a pharmaceutically acceptable salt thereof) and the SSAO inhibitor (such as the compound of (II) or a pharmaceutically acceptable salt thereof) can independently be once daily, twice daily or every other day, for a treatment period of one or more weeks. In some embodiments, the administration comprises administering both compounds daily for a treatment period of one or more weeks. In some embodiments, the administration comprises administering both compounds twice daily for a treatment period of one or more weeks. In some embodiments, the administration comprises administering both compounds every other day for a treatment period of one or more weeks.

In some embodiments, the FXR agonist (such as the compound of Formula (I) or a pharmaceutically acceptable salt thereof) and the SSAO inhibitor (such as the compound of (II) or a pharmaceutically acceptable salt thereof) are administered to the individual once per day for at least seven days, wherein the daily amounts are independently in a range of about 1 mg to about 10 mg, about 1 mg to about 5 mg or about 1 mg to about 3 mg, or about any one of 1, 2, 3, 4, 5, 6, 7, 8, 9, or 10 mg. In some embodiments, both compounds are administered to the individual once per day for at least 14 days, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg. In some embodiments, both compounds are administered to the individual once per day for a period of between one and four weeks, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg.

When administered in combination with a SSAO inhibitor, the FXR agonist and/or the SSAO inhibitor can be administered at doses that are typically administered when either agent is administered alone. Alternatively, as a result of the synergy observed with the combination, the FXR agonist and/or the SSAO inhibitor can be administered at doses that are lower than doses when either agent is administered alone. For instance, in embodiments wherein the FXR agonist is a compound of Formula (I) (e.g., Compound 1) or a pharmaceutically acceptable salt thereof, a therapeutic dose of the compound of Formula (I) to a human patient is typically from about 5 mg to about 15 mg daily administered orally. Hence, in particular embodiments, when administered in combination with a SSAO inhibitor, the compound of Formula (I) or a pharmaceutically acceptable salt thereof can be administered at an oral dose of from about 5 mg to about 15 mg (e.g., 5 mg, 6 mg, 7 mg, 8 mg, 9 mg, 10 mg, 11 mg, 12 mg, 13 mg, 14 mg, or 15 mg) or can be administered at a lower dose. For instance, when administered in combination with a SSAO inhibitor, the compound of Formula (I) or a pharmaceutically acceptable salt thereof can be administered orally at a dose of from about 1 mg to about 15 mg daily, from about 1 mg to about 4.9 mg daily, from about 1 mg to about 4 mg daily, from about 2 mg to about 4 mg daily, or of any of 1, 1.5, 2, 2.5, 3, 3.5, 4, 4.5, 4.9, 5, 6, 7, 8, 9, 10, 11, 12, 13, 14, or 15 mg daily.

In embodiments wherein the SSAO inhibitor is a compound of formula (II) (e.g., Compound 2) or a pharmaceutically acceptable salt thereof, a therapeutic dose of the compound to a human patient is typically from about 4 mg to about 40 mg daily administered orally. In particular embodiments, when administered in combination with a FXR agonist, the compound of formula (II) or a pharmaceutically acceptable salt thereof can be administered at an oral dose of from about 4 mg to about 20 mg (e.g., 4 mg, 5 mg, 6 mg, 8 mg, 10 mg, 15 mg, or 20 mg) or can be administered at a lower dose. For instance, when administered in combination with a FXR agonist, the compound of formula (II) or a pharmaceutically acceptable salt thereof can be administered orally at a dose of from about 1 mg to about 20 mg daily, from about 1 mg to about 3.9 mg daily, from about 1 mg to about 3 mg daily, from about 1.5 mg to about 3.5 mg daily, from about 2 mg to about 3 mg daily, or any of 1, 1.5, 2, 2.5, 3, 3.5, 3.6, 3.8, 3.9, 4, 5, 6, 7, 8, 9, 10, 11, 12, 13, 14, 15, 16, 17, 18, 19, or 20 mg daily.

In particular embodiments wherein the FXR agonist is a compound of formula (I) (e.g., Compound 1) or a pharmaceutically acceptable salt thereof and the SSAO inhibitor is a compound of formula (II) (e.g., Compound 2) or a pharmaceutically acceptable salt thereof, the dose of each individual compound can be administered as set forth above. For instance, in some embodiments, the compound of formula (I) or a pharmaceutically acceptable salt thereof, is administered at a dose from about 1 mg to about 15 mg daily in combination with the compound of formula (II) or a pharmaceutically acceptable salt thereof administered at a dose of from about 1 mg to about 20 mg daily. In some embodiments, the compound of formula (I) or a pharmaceutically acceptable salt thereof is administered at a dose from about 5 mg to about 15 mg daily in combination with the compound of formula (II) or a pharmaceutically acceptable salt thereof administered at a dose of from about 1 mg to about 5 mg daily, from about 1 mg to about 10 mg daily, from about 4 mg to about 20 mg daily, or from about 10 mg to about 20 mg daily. In some embodiments, the compound of formula (I) or a pharmaceutically acceptable salt thereof is administered at a dose from about 1 mg to about 5 mg daily in combination with the compound of formula (II) or a pharmaceutically acceptable salt thereof administered at a dose of from about 1 mg to about 5 mg daily, from about 1 mg to about 10 mg daily, from about 4 mg to about 20 mg daily, or from about 10 mg to about 20 mg daily.

In some embodiments, the amount of the FXR agonist (such as the compound of Formula (I) or a pharmaceutically acceptable salt thereof) and the amount of the SSAO inhibitor (such as the compound of (II) or a pharmaceutically acceptable salt thereof) administered on day 1 of the treatment period are greater than or equal to the amounts administered on all subsequent days of the treatment period. In some embodiments, the amounts administered on day 1 of the treatment period are equal to the amounts administered on all subsequent days of the treatment period.

A compound of Formula (II), or a pharmaceutically acceptable salt thereof, used in accordance with the method described herein can be administered to an individual a once daily dose for a first period of time, followed by a second period of time in which administration of the compound is discontinued, wherein the SSAO inhibitory activity is maintained during both the first and the second period of time. In some embodiments, the first and second periods of time are each one-week periods. For example, provided herein is a method of treatment in an individual for a period of 14 days comprising administering to the individual a once daily dose of a compound of Formula (II), or a pharmaceutically acceptable salt thereof, for a first 7 days, followed by discontinued administration of the compound for the following 7 days, wherein the SSAO inhibitory activity is maintained in the individual during the entire 14-day period. As another example, provided herein is a method of treatment in an individual for a period of four weeks, comprising administering to the individual a once daily dose of a compound of Formula (II), or a pharmaceutically acceptable salt thereof, for a first two weeks, followed by discontinued administration of the compound for the following two weeks, wherein the SSAO inhibitory activity is maintained in the individual during the entire four-week period. In some embodiments, the daily dose is about 10 mg. It is understood that the dosages and dosing regimens disclosed herein are also applicable in a monotherapy for treating NASH using a compound of Formula (II), or a pharmaceutically acceptable salt thereof.

In some embodiments, the administration modulates one or more of the following: a metabolic pathway, bile secretion, retinol metabolism, drug metabolism-cytochrome P450, fat digestion and absorption, glycerolipid metabolism, chemical carcinogenesis, glyceropholipid metabolism, nicotine addiction, linoleic acid metabolism, ABC transporters, metabolism of xenobiotics by cytochrome P450, sphingolipid metabolism, glutathione metabolism, folate biosynthesis, morphine addiction, glycosphingolipid biosynthesis-lacto and neolacto series, arachidonic acid metabolism, tyrosine metabolism, maturity onset diabetes of the young, DNA replication, cholesterol metabolism, drug metabolism—other enzymes, and ether lipid metabolism. In some embodiments,—the administration modulates one or more of the following: a metabolic pathway, retinol metabolism, fat digestion and absorption, glycerolipid metabolism, chemical carcinogenesis, glyceropholipid metabolism, ABC transporters, metabolism of xenobiotics by cytochrome P450, sphingolipid metabolism, glutathione metabolism, folate biosynthesis, and morphine addiction. In some embodiments, the administration modulates expression of one or more of the following: Abcb4, Apoa5, Cyp7a1, Cyp8b1, Nr0b2, and Sic51b.

In some embodiments, administration with a combination of the FXR agonist (such as the compound of Formula (I) or a pharmaceutically acceptable salt thereof) and the SSAO inhibitor (such as the compounds of Formula (II) or a pharmaceutically acceptable salt thereof), to an individual in need thereof, results in differential expression of genes. In some embodiments, administration with the combination results in differential expression of genes as compared to a vehicle control. In some embodiments, administration with the combination results in differential expression of genes associated with lipid metabolism and fatty acid transportation. Genes related to lipid metabolism and/or fatty acid transportation include, but are not limited to, V1d1r, Fabp2, I11r2, Vegfc, Lrp2, Irs2, Vegfa, Lrp1, Irs1, Ppara, S1c27a1, Ld1rap1, Ld1r, Ppargc1a, Rxra, S1c27a5. In some embodiments, administration with the combination results in differential expression of at least 1, at least 2, at least 3, at least 4, at least 5, at least 6, at least 7, at least 8, at least 9, or at least 10 of V1d1r, Fabp2, I11r2, Vegfc, Lrp2, Irs2, Vegfa, Lrp1, Irs1, Ppara, S1c27a1, Ld1rap1, Ld1r, Ppargc1a, Rxra, and S1c27a5, as compared to a vehicle control.

In some embodiments, administration with the combination increases the level of expression of one or more genes related to lipid metabolism and/or fatty acid transportation relative to a vehicle control. In some embodiments, administration with the combination increases the level of expression of at least one gene related to lipid metabolism and/or fatty-acid transportation by between about 1- and about 1.5-fold, between about 1.5- and about 2-fold, between about 2- and about 2.5-fold, between about 2.5- and about 3-fold, between about 3- and about 3.5-fold, or greater than about 3.5-fold, relative to an untreated control. In some embodiments, administration with the combination increases the level of expression of at least one gene related to lipid metabolism and/or fatty acid transportation, wherein the at least one gene related to lipid metabolism and/or fatty acid transportation is selected from Lrp2, Irs2, Vegfa, Lrp1, Irs1, Ppara, S1c27a1, Ld1rap1, Ld1r, Ppargc1a, Rxra, and S1c27a5.

In some embodiments, administration with the combination reduces the level of expression of one or more genes related to lipid metabolism and/or fatty acid transportation. In some embodiments, the level of expression of the one or more genes related to lipid metabolism and/or fatty acid transportation is reduced between about 1- and about 1.5-fold, between about 1.5- and about 2-fold, between about 2- and about 2.5-fold, between about 2.5- and about 3-fold, between about 3- and about 3.5-fold, or greater than about 3.5-fold, relative to an untreated control. In some embodiments, administration with the combination reduces the level of expression of at least one gene related to lipid metabolism and/or fatty acid transportation, wherein the at least one gene related to lipid metabolism and/or fatty acid transportation is selected from V1d1r, Fabp2, I11r2, and Vegfc.

Thus it is understood that methods of treatment detailed herein, in some embodiments, comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) in an individual in need thereof, wherein treatment comprises reducing expression of one or more genes related to lipid metabolism and/or fatty acid transportation. In some embodiments, the methods comprise reducing Fabp2 expression, especially hepatic Fabp2 expression.

In some embodiments, administration with the combination results in differential expression of one or more genes related to lipid metabolism and/or fatty acid transportation as compared to administration with a monotherapy of the FXR agonist or the SSAO inhibitor. Hence, in such embodiments, the FXR agonist potentiates the anti-steatotic effect of the SSAO inhibitor. In some embodiments, administration with the combination increases expression of one or more genes related to lipid metabolism and/or fatty acid transportation as compared to administration with a monotherapy of the FXR agonist. In some embodiments, administration with the combination increases expression of one or more genes related to lipid metabolism and/or fatty acid transportation selected from Irs2, Irs1, Ppara, S1c27a1, Ld1rap1, Ld1r, Ppargc1a, Rxra, and S1c27a5, as compared to administration with a monotherapy of the FXR agonist. In some embodiments, administration with the combination increases expression of one or more genes related to lipid metabolism and/or fatty acid transportation selected from Lrp2, Irs2, Vegfa, Lrp1, Irs1, Ppara, S1c27a1, Ldr1, Ppargc1a, Rxra, and S1c27a5, as compared to administration with a monotherapy of the SSAO inhibitor. In some embodiments, administration with the combination reduces expression of one or more genes related to lipid metabolism and/or fatty acid transportation as compared to administration with a monotherapy of the FXR agonist. In some embodiments, administration with the combination reduces expression of one or more genes related to lipid metabolism and/or fatty acid transportation selected from V1d1r, Fabp2, I11r2, and Vegfc, as compared to administration with a monotherapy of the FXR agonist. In some embodiments, administration with the combination increases expression of one or more genes related to lipid metabolism and/or fatty acid transportation selected from Fabp2, I11r2, and Vegfc, as compared to administration with a monotherapy of the SSAO inhibitor.

Thus it is to be understood that in some embodiments, methods of treatment with a combination of the FXR agonist (such as the compound of Formula (I) or a pharmaceutically acceptable salt thereof) and the SSAO inhibitor (such as the compounds of Formula (II) or a pharmaceutically acceptable salt thereof) as detailed herein comprise treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, wherein treatment comprises differential expression of genes related to lipid metabolism and/or fatty acid transportation such as V1d1r, Fabp2, I11r2, Vegfc, Lrp2, Irs2, Vegfa, Lrp1, Irs1, Ppara, S1c27a1, Ld1rap1, Ld1r, Ppargc1a, Rxra, and S1c27a5. In some embodiments, treatment comprises increasing expression of one or more genes related to lipid metabolism and/or fatty acid transportation selected from Lrp2, Irs2, Vegfa, Lrp1, Irs1, Ppara, S1c27a1, Ld1rap1, Ld1r, Ppargc1a, Rxra, and S1c27a5. In some embodiments, treatment comprises increasing expression of one or more genes related to lipid metabolism and/or fatty acid transportation selected from Irs2, Irs1, Ppara, S1c27a1, Ld1rap1, Ld1r, Ppargc1a, Rxra, and S1c27a5, as compared to administration with a monotherapy of the FXR agonist. In some embodiments, treatment comprises increasing expression of one or more genes related to lipid metabolism and/or fatty acid transportation selected from Lrp2, Irs2, Vegfa, Lrp1, Irs1, Ppara, S1c27a1, Ldr1, Ppargc1a, Rxra, and S1c27a5, as compared to administration with a monotherapy of the SSAO inhibitor. In some embodiments, treatment comprises reducing expression of one or more genes related to lipid metabolism and/or fatty acid transportation selected from V1d1r, Fabp2, I11r2, and Vegfc, as compared to administration with a monotherapy of the FXR agonist. In some embodiments, treatment comprises reducing expression of one or more genes related to lipid metabolism and/or fatty acid transportation selected from Fabp2, I11r2, and Vegfc, as compared to administration with a monotherapy of the SSAO inhibitor.

It is to be understood that recitation of any gene (e.g. Fabp2) as described herein comprises a reference to orthologs from all species, including humans and rodents.

In certain embodiments, the methods of treatment detailed herein comprise treating an individual in need thereof with the combination of the FXR agonist (such as the compound of Formula (I) or a pharmaceutically acceptable salt thereof) and the SSAO inhibitor (such as the compound of (II) or a pharmaceutically acceptable salt thereof) in a ratio of about 3 units of FXR agonist to about 25 units of SSAO inhibitor by weight.

Also provided herein are combinations of the FXR agonist (such as the compound of Formula (I) or a pharmaceutically acceptable salt thereof) and the SSAO inhibitor (such as the compounds of Formula (II) or a pharmaceutically acceptable salt thereof) for use in treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

Also provided herein are uses of the combinations of the FXR agonist (such as the compound of Formula (I) or a pharmaceutically acceptable salt thereof) and the SSAO inhibitor (such as the compounds of Formula (II) or a pharmaceutically acceptable salt thereof) for manufacture of a medicament for treating a liver disorder such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH) an individual in need thereof, using the methods as described herein.

### Articles of Manufacture and Kits

The present disclosure further provides articles of manufacture comprising a compound described herein, or a salt thereof, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The present disclosure further provides kits for carrying out the methods of the present disclosure, which comprises at least two compounds described herein, or a pharmaceutically acceptable salt thereof, or a composition comprising a compound described herein, or a pharmaceutically acceptable salt thereof. The kits may employ any of the compounds disclosed herein or a pharmaceutically acceptable salt thereof. In some embodiments, the kit employs an FXR agonist (such as the compound of Formula (I) or a pharmaceutically acceptable salt thereof) and an SSAO inhibitor (such as the compound of (II) or a pharmaceutically acceptable salt thereof) described herein. The kits may be used for any one or more of the uses described herein, and, accordingly, may contain instructions for the treatment as described herein.

Kits generally comprise suitable packaging. The kits may comprise one or more containers comprising any compound described herein or a pharmaceutically acceptable salt thereof. Each component can be packaged in separate containers or some components can be combined in one container where cross-reactivity and shelf life permit. In some embodiments, the kit includes a container comprising the FXR agonist (such as the compound of Formula (I) or a pharmaceutically acceptable salt thereof) and the SSAO inhibitor (such as the compound of (II) or a pharmaceutically acceptable salt thereof). In other embodiments, the kit includes a first container comprising FXR agonist (such as the compound of Formula (I) or a pharmaceutically acceptable salt thereof) and a second container comprising the SSAO inhibitor (such as the compound of (II) or a pharmaceutically acceptable salt thereof).

The kits may be in unit dosage forms, bulk packages (e.g., multi-dose packages) or sub-unit doses. For example, kits may be provided that contain sufficient dosages of a compound as disclosed herein, or a pharmaceutically acceptable salt thereof, and/or an additional pharmaceutically active compound useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### ENUMERATED EMBODIMENTS

Embodiment 1. A method of treating a liver disorder in a patient in need thereof, comprising administering to the patient a Farnesoid X Receptor (FXR) agonist and a Semicarbazide-Sensitive Amine Oxidase (SSAO) inhibitor, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Embodiment 2. The method of embodiment 1, wherein the FXR agonist is obeticholic acid, cilofexor, tropifexor, EYP001 (Vonafexor, proposed INN), MET409 (Metacrine), or EDP-305 (by Enanta).

Embodiment 3. The method of embodiment 1 or 2, wherein the SSAO inhibitor is PXS-4728A (BI-1467335).

Embodiment 4. The method of embodiment 1, wherein the FXR agonist is a compound of formula (I)

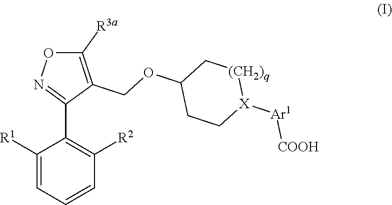

wherein:
q is 1 or 2;
R¹ is chloro, fluoro, or trifluoromethoxy;
R² is hydrogen, chloro, fluoro, or trifluoromethoxy;
R^{3a} is trifluoromethyl, cyclopropyl, or isopropyl;

### X is CH or N,

provided that when X is CH, q is 1; and
Ar¹ is indolyl, benzothienyl, naphthyl, phenyl, benzoisothiazolyl, indazolyl, or pyridinyl, each of which is optionally substituted with methyl or phenyl,
or a pharmaceutically acceptable salt thereof.
Embodiment 5. The method of embodiment 4, wherein:
R¹ is chloro or trifluoromethoxy; and
R² is hydrogen or chloro.
Embodiment 6. The method of embodiment 4 or 5, wherein:
R^{3a} is cyclopropyl or isopropyl.
Embodiment 7. The method of any one of embodiments 4 to 6, wherein:
Ar¹ is 5-benzothienyl, 6-benzothienyl, 5-indolyl, 6-indolyl, or 4-phenyl, each of which is optionally substituted with methyl.
Embodiment 8. The method of any one of embodiments 4 to 7, wherein:
q is 1; and

### X is N.

Embodiment 9. The method of embodiments 1 or 4, wherein the FXR agonist is:

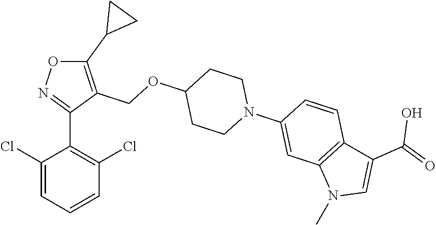 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof.
Embodiment 10. The method of any one of embodiments 1, 2, and 4 to 9, wherein the SSAO inhibitor is a compound of formula (II)

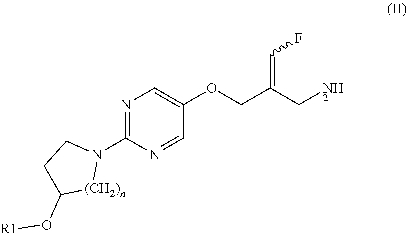

wherein:
n is 1 or 2; and

### R1 is H or —CH₃,

or a pharmaceutically acceptable salt thereof.
Embodiment 11. The method of embodiment 10, wherein the SSAO inhibitor is a compound of formula (IIa)

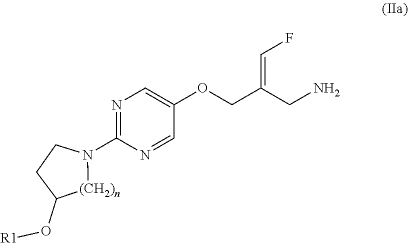

wherein:
n is 1 or 2; and

### R1 is H or —CH₃,

or a pharmaceutically acceptable salt thereof.
Embodiment 12. The method of embodiment 10 or 11, wherein n is 2.
Embodiment 13. The method of any one of embodiments 10 to 12, wherein R1 is CH₃.
Embodiment 14. The method of any one of embodiments 1, 2, and 4 to 9, wherein the SSAO inhibitor is:

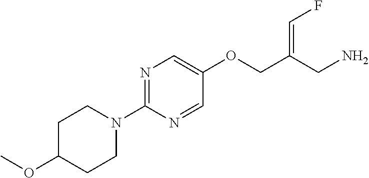 [SMILES: COC1CCN(c2ncc(OC/C(=C/F)CN)cn2)CC1]

or a pharmaceutically acceptable salt thereof.
Embodiment 15. The method of any one of embodiments 1 to 14, wherein the FXR agonist and the SSAO inhibitor are administered simultaneously.
Embodiment 16. The method of any one of embodiments 1 to 14, wherein the FXR agonist and the SSAO inhibitor are administered sequentially.
Embodiment 17. The method of any one of embodiments 1 to 16, wherein the administration does not result in pruritus in the patient at a severity of Grade 2 or more.
Embodiment 18. The method of any one of embodiments 1 to 17, wherein the administration does not result in pruritus in the patient at a severity of Grade 1 or more.
Embodiment 19. The method of any one of embodiments 1 to 18, wherein the administration does not result in pruritus in the patient.
Embodiment 20. The method of any one of embodiments 1 to 19, wherein the patient also has diabetes mellitus and/or a cardiovascular disorder.
Embodiment 21. The method of any one of embodiments 1 to 20, wherein the treatment period is the remaining lifespan of the patient.
Embodiment 22. The method of any one of embodiments 1 to 21, wherein the method does not comprise administering an antihistamine, an immunosuppressant, a steroid, rifampicin, an opioid antagonist, or a selective serotonin reuptake inhibitor (SSRI).
Embodiment 23. The method of any one of embodiments 1 to 22, wherein the FXR agonist is administered once daily or twice daily.
Embodiment 24. The method of any one of embodiments 1 to 23, wherein the SSAO inhibitor is administered once daily or twice daily.
Embodiment 25. The method of any one of embodiments 1 to 24, wherein the administration comprises administering the FXR agonist daily for a treatment period of one or more weeks.
Embodiment 26. The method of any one of embodiments 1 to 25, wherein the administration comprises administering the SSAO inhibitor daily for a treatment period of one or more weeks.
Embodiment 27. The method of any one of embodiments 1 to 26, wherein the liver disorder is selected from the group consisting of non-alcoholic fatty liver disease (NAFLD) and non-alcoholic steatohepatitis (NASH).
Embodiment 28. The method of any one of embodiments 1 to 26, wherein the liver disorder is non-alcoholic steatohepatitis.
Embodiment 29. A pharmaceutical composition comprising an effective amount of an FXR agonist, a therapeutically effective amount of an SSAO inhibitor, and a pharmaceutically acceptable carrier, diluent, excipient, or a combination of any of the foregoing.
Embodiment 30. A dosage form comprising a therapeutically effective amount of an FXR agonist and a therapeutically effective amount of an SSAO inhibitor.
Embodiment 31. A kit comprising a container comprising an FXR agonist and an SSAO inhibitor.
Embodiment 32. A kit comprising a first container comprising an FXR agonist and a second container comprising an SSAO inhibitor.
Embodiment 33. The pharmaceutical composition of embodiment 29, the dosage form of embodiment 30, the kit of embodiment 31 or 32, wherein the FXR agonist is

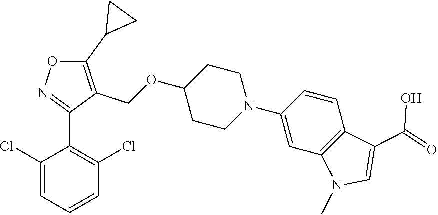 [SMILES: Cn1cc(C(=O)O)c2ccc(N3CCC(OCc4c(-c5c(Cl)cccc5Cl)noc4C4CC4)CC3)cc21]

or a pharmaceutically acceptable salt thereof, and the SSAO inhibitor is:

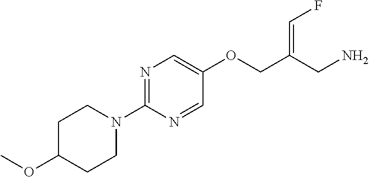 [SMILES: COC1CCN(c2ncc(OC/C(=C/F)CN)cn2)CC1]

or a pharmaceutically acceptable salt thereof.

### EXAMPLES

### Example 1: In Vitro Metabolic Stability

The rate of hepatic metabolism of Compound 1 was assessed in cryopreserved hepatocytes to determine the in vitro half-life of the compound. 1 μM of Compound 1 was mixed with preconditioned mouse, rat, dog, monkey, or human hepatocytes (0.5×10⁶ cells/mL) and allowed to incubate at 37° C. for 2 hours, with samples collected at several time points and assayed for Compound 1. In vitro half-life values were determined and scaled to predict hepatic clearance (CL_{pred}) and hepatic extraction using the well-stirred liver model with no correction for plasma protein as described in Obach et al., The Prediction of Human Pharmacokinetic Parameters from Preclinical and In Vitro Metabolism Data, J. of Pharmacology and Experimental Therapeutics, vol. 283, no. 1, pp. 46-58 (1997). Results are shown in Table 1, which demonstrate that Compound 1 was moderately metabolized in hepatocytes of all tested species.

TABLE 1
In Vitro metabolic stability of Compound 1

| | | In vitro | |
|---|---|---|---|
| | | Metabolic | Hepatic |
| | t_{1/2} | CL_{pred} | Extraction |
| Species | (min) | (L/h/kg) | (%) |
| Mouse | 43.6 ± 2.83 | 4.36 ± 0.06 | 80.7 ± 1.02 |
| Sprague- | 131 ± 4.11 | 1.57 ± 0.03 | 47.3 ± 0.78 |
| Dawley Rat | | | |
| Beagle Dog | 126 ± 15.5 | 1.32 ± 0.05 | 71.0 ± 2.49 |
| Cynomolgus | 63.4 ± 0.78 | 1.68 ± 0.01 | 64.4 ± 0.28 |
| Monkey | | | |
| Human | 84.1 ± 6.48 | 0.83 ± 0.22 | 67.0 ± 1.73 |

### Example 2: In Vitro OATP Transport Assay

A polarized monolayer of MDCK-II cells grown on a permeable support was used to test the ability of organic-anion-transporting polypeptide (OATP) 1B1 or OATP 1B3 to transport Compound 1 across the lipid bilayer and into the cells. The MDCK-II cells were transfected one of (1) a vector to express OATP 1B1, (2) a vector to express OATP 1B3, or (3) a control vector. Expression was induced in the cells before culturing the cells at 37° C. in 5% CO₂ atmosphere. After inducing expression, the cells were treated with 1 μM, 3 μM, and 10 μM Compound 1, or 3 μM Compound 1 and 100 μM rifampin. Cellular uptake of Compound 1 was then measured. Results from this experiment demonstrated that Compound 1 is not an OATP 1B1 or OATP 1B3 substrate.

### Example 3: Pharmacokinetics Assay

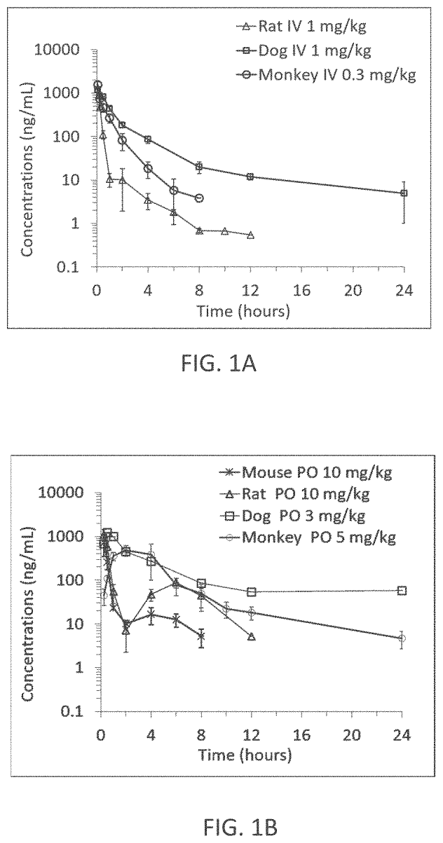

Compound 1 was administered to Sprague-Dawley (SD) rats intravenously at 1 mg/kg (n=3) or orally at 10 mg/kg (n=3), to beagle dogs intravenously at 1 mg/kg (n=3) or orally at 3 mg/kg (n=3), to cynomolgus monkeys intravenously at 0.3 mg/kg (n=6) or orally at 5 mg/kg (n=6), and to mice orally at 5 mg/kg (n=9). Compound 1 for oral administration to SD rats was formulated in a vehicle containing 10% DMSO, 10% Cremophor-EL, and 80% aqueous solution (10% 2-hydroxypropyl-β-cyclodextrin). Compound 1 for oral administration to beagle dogs was formulated with an aqueous solution containing 1% carboxymethyl cellulose, 0.25% Tween-80, and 0.05% antifoam. Compound 1 for oral administration to cynomolgus monkeys was formulated with 10% Solutol, 20% PEG400, 0.5% Tween-80 and 69.5% deionized water. Serial blood samples were collected, and plasma concentrations of the Compound 1 were measured. Results are shown in FIG. 1A (IV administration) and FIG. 1B (oral administration), and in Table 2. The results demonstrate that Compound 1 has low to moderate clearance in vivo. The volume of distribution (V_{dss}) of Compound 1 is greater than the volume of total body water (0.70 L/kg) in rat and dog. Smaller V_{dss} in monkeys is correlated with higher plasma protein binding.

TABLE 2
Pharmacokinetic parameters of Compound 1

| | CL | V_{dss} | IV Terminal | Oral |
|---|---|---|---|---|
| Species | (L/h/kg) | (L/kg) | t_{1/2} (h) | Bioavailability (%) |
| Sprague- | 2.55 | 1.31 | 2.45 | 21 |
| Dawley Rat | | | | |
| Beagle Dog | 0.54 | 1.92 | 5.67 | 82 |
| Cynomolgus | 0.30 | 0.6 | 1.32 | 18 |
| Monkey | | | | |

### Example 4: Tissue Distribution of Compound 1

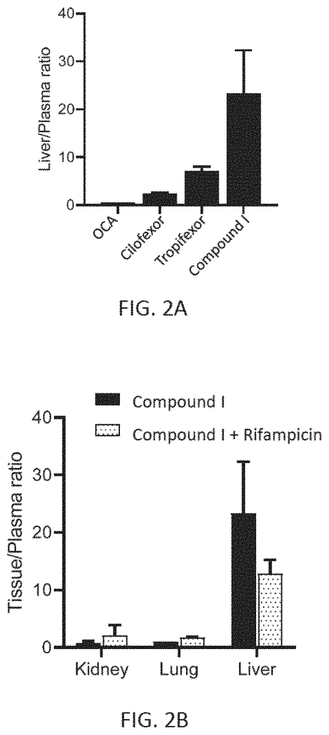

Tissue distribution of Compound 1 administered to rats was determined and compared to distribution other Farnesoid X Receptor (FXR) agonists cilofexor, tropifexor, and obeticholic acid (OCA). The tested compounds were administered to SD rats (n=3 per compound) by way of 30 minute intravenous infusion at 2 mg/kg. Blood, liver, kidney, and lung tissue samples were collected from the rats to determine a tissue/plasma ratio. The liver tissue/plasma ratio for the compounds is shown in FIG. 2A, which demonstrates that substantially more of Compound 1 localizes to the liver tissue compared to the other tested compounds. Co-administration of Compound 1 with 100 μM rifampin does not result in a significant change in distribution of Compound 1 to the liver (FIG. 2B). These results collectively demonstrated that Compound 1 is preferentially distributed to the liver and exhibited high liver/plasma ratio in rodent species, approximately 3 to 20-fld higher than other FXR agonists being studied for the treatment of NASH (cilofexor, tropifexor, and OCA).

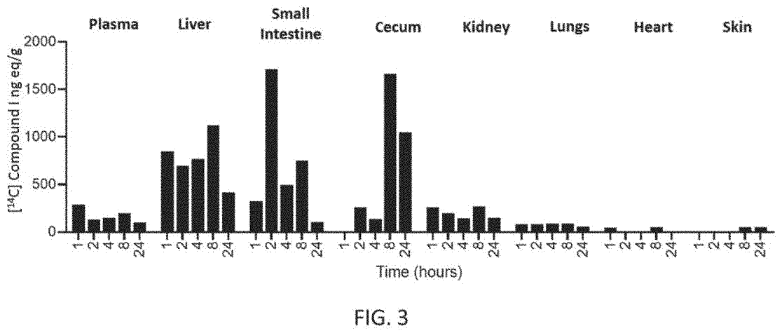

Radiolabeled (¹⁴C) Compound 1 was also administered to Long-Evans rats at an oral dose of 5 mg/kg (100 μCi/kg). Plasma, liver, small intestine, cecum, kidney, lung, heart and skin tissue samples were collected up to 168 hours, and the amount of radioactive material at various time points was measured. Results are shown in FIG. 3. Liver, small intestine, and cecum had the most radioactive material.

### Example 5: Metabolism of Compound 1

Radiolabeled (¹⁴C) Compound 1 was administered to bile duct intact or cannulated SD rats orally at 5 mg/kg or intravenously at 2 mg/kg (n=3 for each of the four cohorts) for a total radioactive dose of 100 μCi/kg. Blood, bile, feces, and urine samples were collected from each rat for up to 168 hours. Compound 1 was metabolized into an acyl glucuronide metabolite prior to biliary excretion, which was determined as the major elimination pathway for the compound.

### Example 6: Pharmacokinetics/Pharmacodynamics Profile

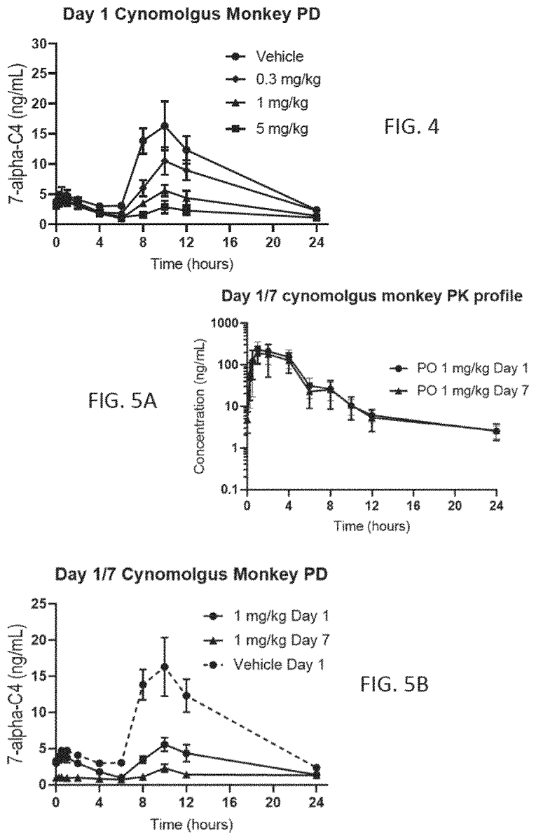

Pharmacokinetics/pharmacodynamics (PK/PD) profiles for cynomolgus monkeys was determined by administering an oral dose of Compound 1 suspension at doses of 0 (vehicle), 0.3, 1, or 5 mg/kg, and collecting blood samples for up to 24 hours. The pharmacodynamics were measured as a function of 7-alpha-hydroxy-4-cholesten-3-one (7AC4) reduction (FIG. 4), as quantified by LC-MS/MS. Pharmacokinetics data is presented in Table 3, and were determined by non-compartmental analysis.

TABLE 3
Pharmacokinetic parameters of Compound 1

| | PK Parameters | | |
|---|---|---|---|
| Compound 1 | AUC₀₋₂₄ | Cₘₐₓ | Tₘₐₓ |
| dose | (ng*hr/mL) | (ng/mL) | (hr) |
| 0.3 mg/kg | 196 ± 64 | 58.8 ± 30.2 | 2.17 ± 1.47 |
| 1 mg/kg | 1000 ± 419 | 257 ± 124 | 1.83 ± 1.17 |
| 5 mg/kg | 2720 ± 1500 | 709 ± 458 | 2.25 ± 1.47 |

Compound 1 was also orally administered at 1 mg/kg for 7 consecutive days to cynomolgus monkeys (n=6) to determine the PK/PD profile following multiple doses. Results of this study are shown in FIG. 5A (PK profile) and FIG. 5B (PD profile) and Table 4, and demonstrate that the plasma exposure of Compound 1 was comparable on day 1 and day 7 and that sustained suppression of the pharmacodynamics biomarker 7AC4 was achieved after repeated oral dosing.

TABLE 4
Pharmacokinetic parameters of Compound 1

| PK | Cₘₐₓ | AUC₀₋₂₄ | Tₘₐₓ |
|---|---|---|---|
| Parameters | (ng/mL) | (ng*hr/mL) | (hr) |
| Day 1 | 257 ± 124 | 1000 ± 419 | 1.83 ± 1.17 |
| Day 7 | 221 ± 121 | 858 ± 425 | 1.25 ± 0.61 |

### Example 7: Mechanism of Action

C57BL/6 mice were administered a single oral dose of 10 mg/kg Compound 1 (n=6), 30 mg/kg OCA (n=6), or a vehicle control (n=6), and tissue RNA samples were collected 6 hours after dose administration. The RNA was analyzed by RT-qPCR and RNAseq.

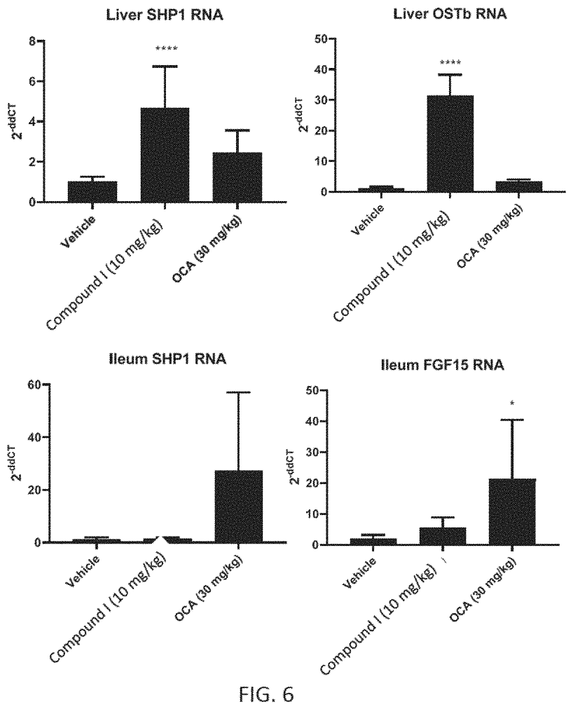

For RT-qPCR, gene-specific primers were used to quantitate FXR-regulated gene expression in liver and ileum using the 2-ddCT method. Results are shown in FIG. 6 (data presented as mean±SEM; **** indicates p<0.0001 and * indicates p<0.05 versus vehicle, with statistics determined by one-way ANOVA followed by Tukey). This data indicates that Compound 1 preferentially induces FXR-specific genes in the liver of mice.

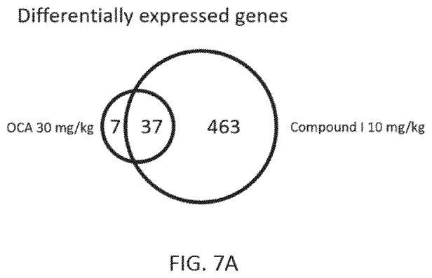

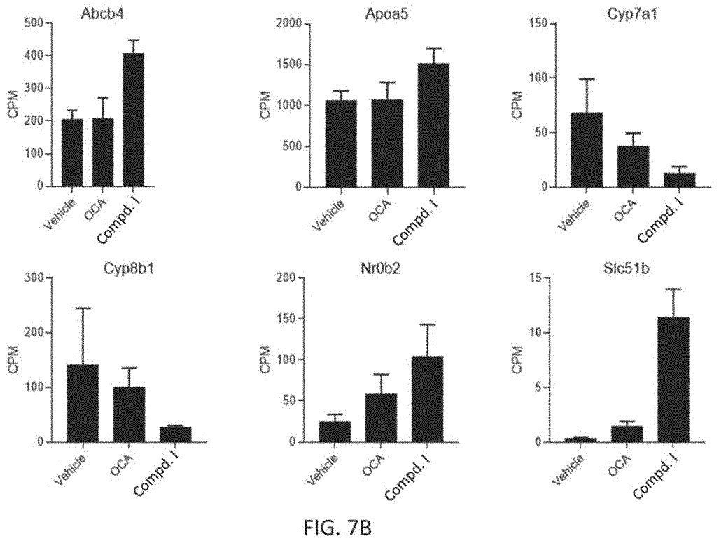

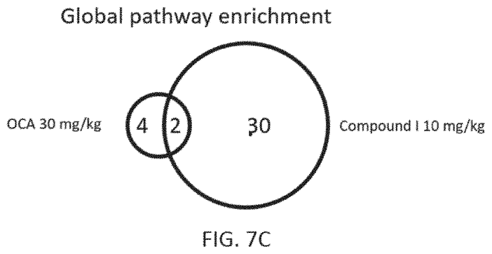

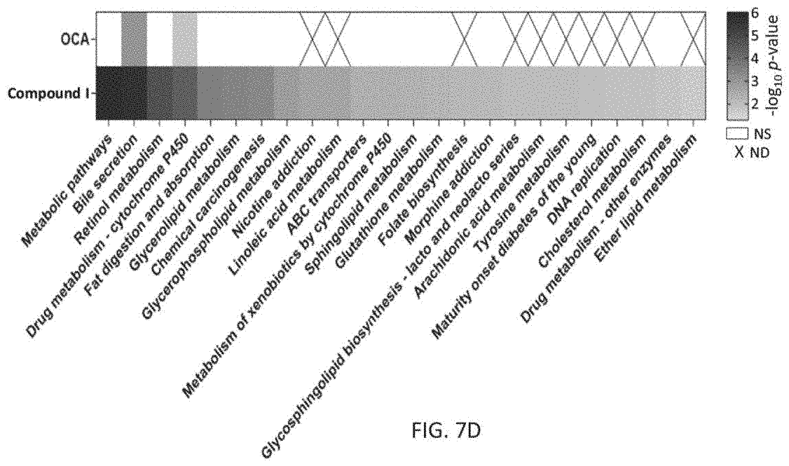

For RNAseq analysis, mRNA was extracted from total liver and sequenced using standard Illumina library preparation and sequencing protocols. Differentially expressed genes (DEG) were determined using RSEM and edgeR software packages and analyzed using Advaita Bio's iPathwayGuide software. Results are shown in FIG. 7A-7D, which indicate that Compound 1 modulates a significantly higher number of genes and metabolic pathways relevant to NASH compared to OCA. FIG. 7A shows that administration of Compound 1 modulates expression of 500 NASH-related genes, OCA modulates expression of 44 NASH-related genes, including 37 common NAS-related genes modulated by both Compound 1 and OCA, relative to vehicle control (fold change ≥1.5; q-value <0.05). FIG. 7B shows average expression levels (as shown by CPM value) of select FXR-related genes in vehicle, OCA, and Compound 1 treated mice. FIG. 7C shows that administration of Compound 1 causes enrichment of 32 global pathways and that administration of OCA causes enrichment of 6 global pathways, including 2 common global pathways to both Compound 1 and OCA administration. FIG. 7D shows the 25 pathways most statistically enriched upon Compound 1 administration, and compares the enrichment of those pathways to the enrichment upon OCA administration. Overall, RNAseq analysis of livers from mice treated with Compound 1 showed a more robust modulation of FXR-related genes and metabolic pathways relevant to non-alcoholic fatty liver disease compared to OCA treatment.

### Example 8: Clinical Study

First Study. Heathy human volunteer subjects were orally dosed on a daily basis with Compound 1 at 5 mg (n=9), 75 mg (n=9), 200 mg, or 400 mg (n=18), or received a placebo (n=12) for 14 days. During this study, no incidences of pruritus were observed.

Second Study. Compound 1 was administered daily for 7 days at oral doses of 25 mg (n=11), 75 mg (n=10), or 150 mg (n=10), or received a placebo (n=5) to human subjects. 7-alpha-hydroxy-4-cholesten-3-one (7AC4) levels in the patients were periodically measured, as shown in Table 5, which indicated that levels were suppressed by Compound 1. In a separate study published by an independent group, FXR agonist MET409 (Metacrine) was reportedly administered daily to healthy human volunteers at doses of 20 mg 40 mg, 50 mg, 80 mg, 100 mg, or 150 mg, and 7AC4 levels measured as shown in Table 5. See Chen et al., MET409, an Optimized Sustained FXR Agonist, Was Safe and Well-Tolerated in a 14-Day Phase 1 Study in Healthy Subjects, The International Liver Congress, Vienna, Austria, Apr. 10-14, 2019. While pruritus was observed in subjects receiving MET409 at doses of 100 mg or greater, no pruritus was observed for subjects taking the highest doses of Compound 1. Other FXR agonists, such as cilofexor, tropifexor, OCA, EDP-305 (Enanta) are all known to result in pruritus in longer term studies.

TABLE 5
Comparison of MET409 and Compound 1

| | MET409 | | | | | |
|---|---|---|---|---|---|---|
| | 50 mg | 80 mg | 100 mg | Compound 1 | | |
| Parameters | MET409 | MET409 | MET409 | 25 mg | 75 mg | 150 mg |
| AUC | 6404 | 12479 | 16519 | 645 | 1480 | 2164 |
| ng*h/ml | | | | | | |
| % 7AC4 | 85% | 96% | 99% | 75% | 82% | 93% |
| suppression | | | | | | |
| at nadir | | | | | | |
| AUC/% | 75 | 130 | 166 | 8.6 | 18 | 23 |
| 7AC4 ratio | | | | | | |
| Pruritus | No | No | Yes | No | No | No |

### Example 9: Mouse Model of NASH

The effect of Compound 1 on NASH was assessed using a mouse model, in which NASH is induced by a high fat diet in combination with CCl₄ administration.

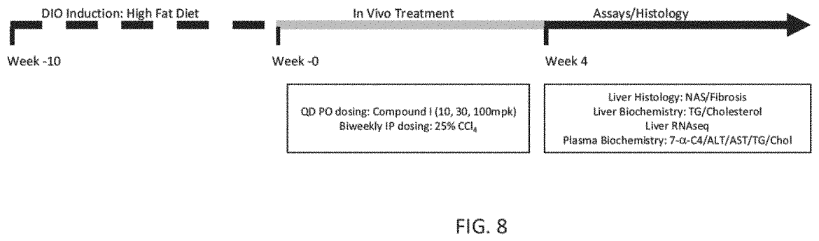

Mice C57/BL6J mice were fed a high fat diet (D12492, Research Diet, fat/protein/carbohydrate 60/20/20 Kcal %, 10 w) to induce obesity (>36 g mouse) prior to daily oral Compound 1 and biweekly intraperitoneal carbon tetrachloride (CCl₄) treatment for four weeks. FIG. 8. Compound 1 was administered at a dose of 10, 30, and 100 mg/kg.

Following 28 days of Compound 1 dosing, serum lipids, serum transaminases and liver lipids were analyzed. Hematoxylin & Eosin (H&E) and Sirius Red histological staining of liver tissue was used to quantitate NAFLD activity score (NAS), steatosis, ballooning, inflammation and fibrosis. Plasma 7-alpha-hydroxy-4-cholesten-3-one (7AC4) was measured as a biomarker of FXR activation. Gene expression of RNA was analyzed by RT-qPCR and RNAseq.

Nonalcoholic Fatty Liver Disease Activity Score (NAS) is a composite score used to assess NASH. NAS is calculated based upon liver steatosis, inflammation, and ballooning and was determined by analysis of liver tissue histology using H&E stain. Specifically, inflammation score was calculated based upon H&E staining: Score 0, none; 1, <2 foci per 200× field; 2, 2-4 foci per 200× field; 3, >4 foci per 200× field. Steatosis score was calculated by H&E staining as follows: Score 0, <5%; 1,5-33%; 2, >33-66%; 3, >66%). Hepatocellular ballooning is a form of liver cell injury associated with cell swelling and is also measured by H&E stained liver sections. The ballooning score is calculated as follows: 0-no hepatocyte ballooning; 1-few ballooning hepatocytes; 2-many hepatocytes with prominent ballooning.

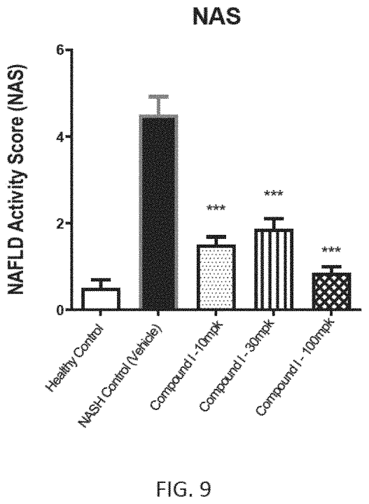

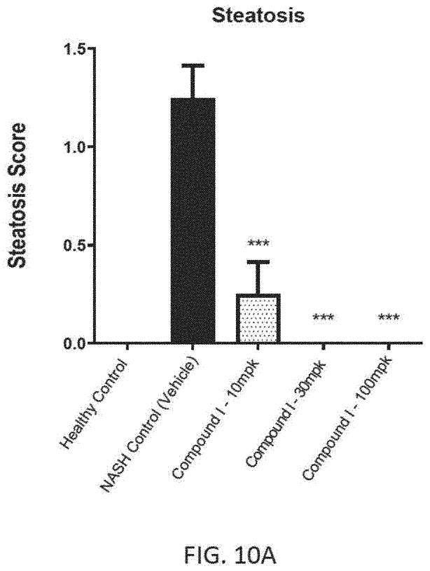

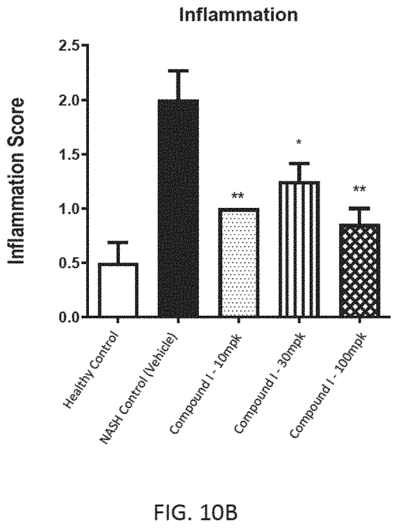

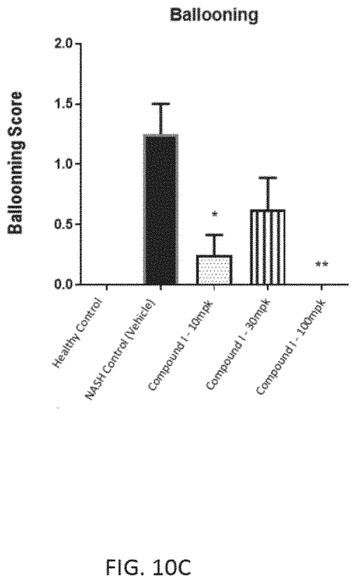

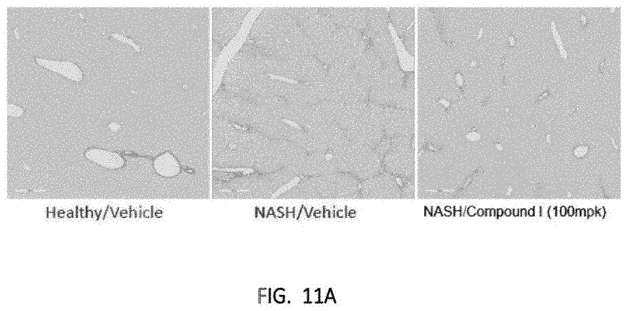

As shown in FIG. 9, mice treated with 10, 30, or 100 mg/kg Compound 1 had a significantly lower NAS score as compared to untreated NASH mice. Treatment with Compound 1 also significantly reduced steatosis, inflammation and ballooning compared to untreated NASH mice. FIG. 10A-C.

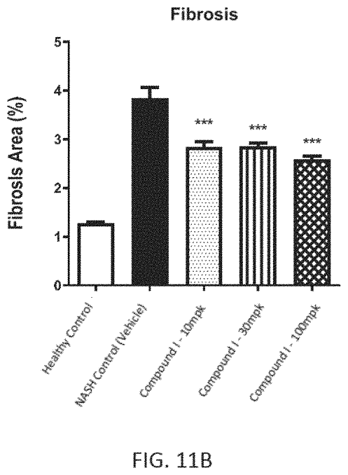

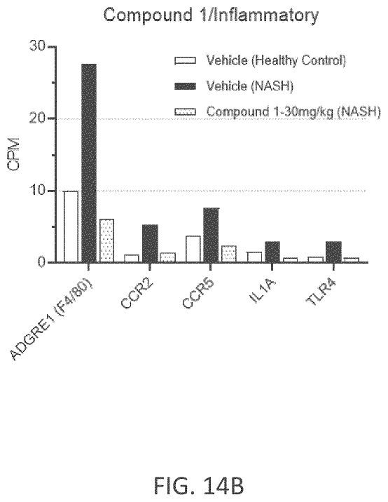

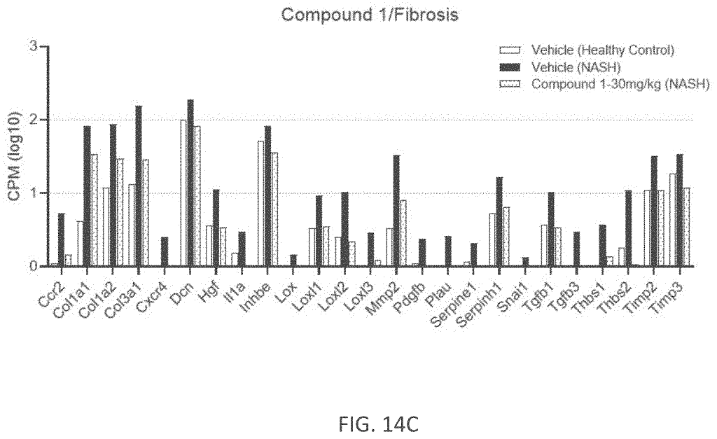

Liver fibrosis was quantified by histological analysis of the percentage of Sirius Red-positive liver sections. FIG. 11A shows representative histology for healthy mice, NASH mice, and NASH mice treated with Compound 1 at 100 mg/kg. FIG. 11B shows quantification of the fibrosis area of mice treated with Compound 1. Treatment with 10, 30 or 100 mg/kg Compound 1 resulted in statistically significant reduced fibrosis compared to untreated NASH control. As shown in FIG. 14A, Compound 1 administered at 10, 30, or 100 mg/kg resulted in decreased collagen, type 1, alpha 1 expression in the liver as compared to control NASH mice.

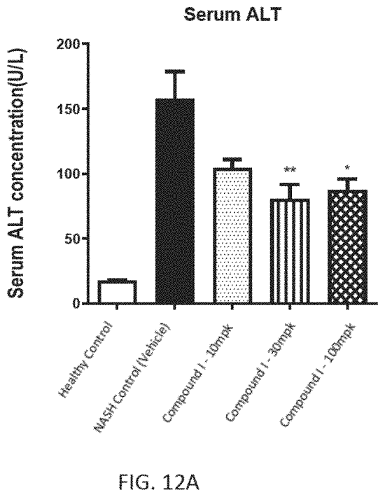

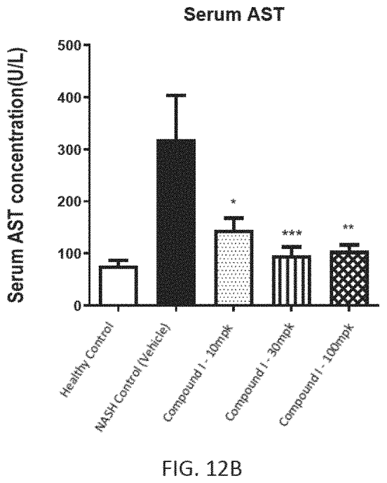

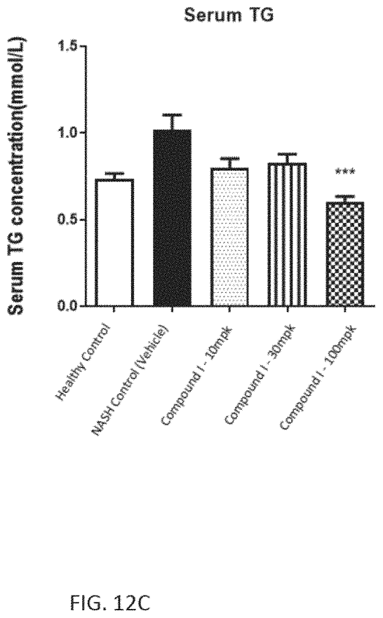

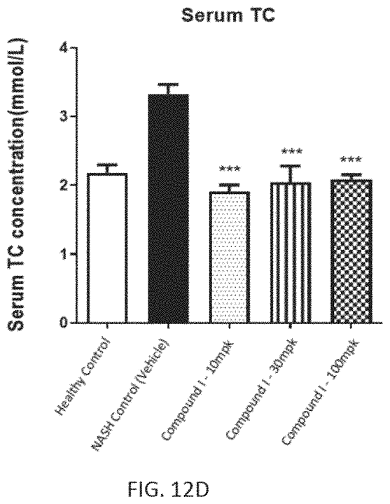

After treatment, serum was analyzed for alanine amino transferase (ALT), aspartate amino transferase (AST), triglyceride, and total cholesterol levels. As shown in FIG. 12A and FIG. 12B serum ALT and AST levels were reduced in mice treated with Compound 1. FIG. 12C shows a statically significant reduction in serum triglyceride concentration in mice treated with 100 mg/kg Compound 1. FIG. 12D shows statistically significant reduction of total cholesterol level in mice treated with 10, 30, and 100 mg/kg Compound 1.

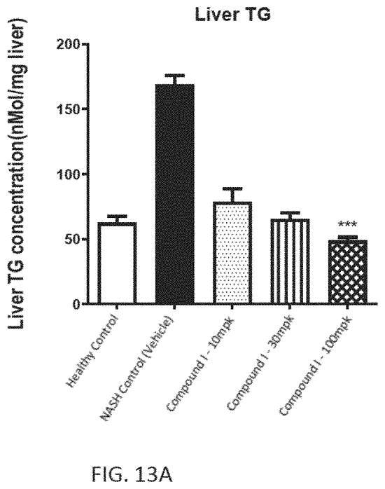

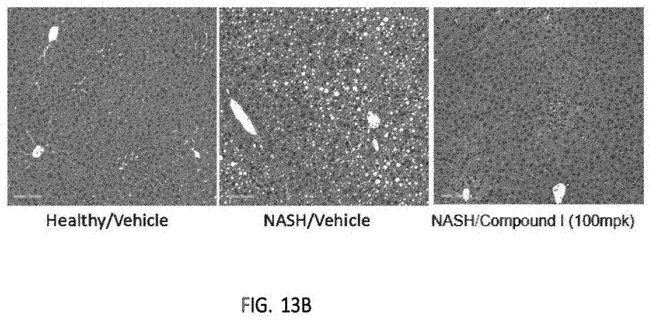

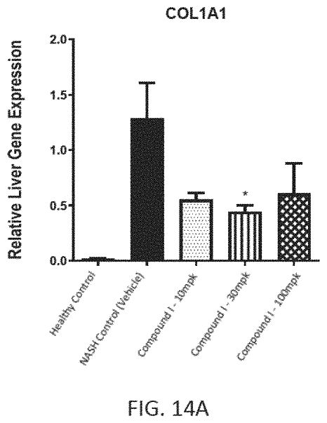

Liver triglycerides were measured from liver tissue using a biochemical analyzer (Hitachi-700). FIG. 13A shows the concentration of liver triglycerides in control mice or mice treated with 10, 30, or 100 mg/kg Compound 1. Mice treated with 100 mg/kg Compound 1 showed statistically significant reduced triglyceride levels. FIG. 13B shows a representative histology section.

The effect of Compound 1 on gene expression was analyzed using RT-qPCR or RNA-seq of liver samples (FIG. 14A-C and Table 6). Table 6 shows the effect of Compound 1 on FXR-regulated gene expression in the liver. The expression level of each indicated gene (as defined by gene count per million (CPM) value) after treatment with Compound 1 was divided by the expression level of that gene in vehicle treated animals to determine the activity of Compound 1 relative to vehicle.

TABLE 6
Expression of FXR-target, inflammatory, and fibrosis genes

| Gene | Compound 1 (30 mg/kg) Relative to Vehicle |
|---|---|
| SHP | 4.6 |
| BSEP | 5.1 |
| OST-B | 135.7 |
| CYP7A1 | 0.02 |
| CYP8B1 | 0.007 |

EC₅₀ concentration of Compound 1 for FXR was determined by a fluorescence-based FXR coactivation assay. Half-log serial dilutions of Compound 1 or OCA (obeticholic acid, a known FXR agonist) (10 μM-3 nM) were incubated with human FXR ligand binding domain produced in Sf9 insect cells, labeled coactivator SRC-1 peptide and TR-FRET Coregulator Buffer G for 1 h at 25° C. TGR5 activity was measured using a cell-based cAMP assay. See Kawamata et al JBC 278 (11)935-440 (2003). Half-log serial dilutions of Compound 1 or OCA (10 μM-3 nM) were added to Chinese Hamster Ovary cells expressing recombinant human TGR5. After 30 min at RT, cAMP was measured using an HTRF readout. EC₅₀ values for FXR-regulated gene expression were determined using a cell-based RNA assay. Half-log serial dilutions of Compound 1 or OCA (3 μM-3 nM) were added to human HuH7 hepatoma cells. After 11 h at 37° C., RNA was isolated and analyzed by RT-qPCR using primers to FXR-related genes: small heterodimer partner (SHP), bile salt export pump (BSEP) and fibroblast growth factor 19 (FGF-19).

As shown in Table 7, Compound 1 is a potent and selective FXR agonist.

TABLE 7
EC₅₀ of Compound 1

| | EC₅₀ of | OCA |
|---|---|---|
| Assay | Compound 1 (nM) | EC₅₀ (nM) |
| FXR Agonist | 57 | 73 |
| TGR5 Agonist | >10,000 | 770 |
| SHP Gene induction/HuH7 | 50 | 200 |
| BSEP Gene induction/HuH7 | 40 | 200 |
| FGF-19 Gene | 40 | 130 |
| Induction/HuH7 | | |

In summary, Compound 1 is a potent and selective FXR agonist. Compound 1 reduced expression of inflammatory and fibrosis related genes and strongly suppressed liver steatosis, inflammation, ballooning, and fibrosis in a mouse model of NASH.

### Example 10

### Background

Semicarbazide-sensitive amine oxidase (SSAO) contributes to non-alcoholic steatohepatitis (NASH) by increasing oxidative stress through deamination of primary amines (e.g., methylamine, MMA) to aldehyde, ammonium, and H₂O₂ and by recruitment of inflammatory cells to the liver, exacerbating hepatic inflammation and injury. SSAO levels are elevated in NASH and correlate with fibrosis stage. Compound 2 is a selective, covalent SSAO inhibitor that decreases liver inflammation and fibrosis in a rat model of NASH. A single-ascending dose clinical trial of Compound 2 was performed.

The compounds described herein may be obtained by the methods described in WO 2018/028517, which is incorporated herein by reference in its entirety and specifically with respect to the methods of making the compounds detailed herein.

### Methods

Four groups of 8 healthy participants were randomized to receive Compound 2 capsule or matching placebo in a 3:1 ratio. Plasma levels of Compound 2 and PD biomarkers were determined at pre-dose and various time points post-dose. SSAO inhibition was determined by measuring relative reductions in plasma H₂O₂ generation after addition of an exogenous substrate (benzylamine). Endogenous methylamine (MMA) levels, predicted to increase upon SSAO inhibition, were measured in plasma. Safety was assessed for 7 (±3) days after dosing.

Plasma samples for Compound 2 concentration and SSAO activity determination were collected at 0.25, 0.5, 1, 2, 3, 4, 6, 8, 10, 12, 24, 48 (SSAO activity only), and 168 (SSAO activity only) hours after administration of a single dose of study medication (placebo or compound). Plasma PK parameters were determined by non-compartmental analysis. SSAO activity was assessed by measuring hydrogen peroxide (H₂O₂) generation levels in plasma samples from placebo and active Compound 2 recipients. Percent change in total amine oxidase activity was determined relative to the corresponding pre-dose (baseline) samples.

SSAO-specific amine oxidase levels in plasma were determined using a kinetic-based assay essentially as described previously (Schilter et al). Endogenous monoamine oxidases A and B were inhibited by adding pargyline to plasma samples prior to measuring H₂O₂ generation levels in placebo and active recipients. Maximum inhibition was defined by pre-dose (baseline) samples additionally treated with a high dose of Compound 2 and percent changes in SSAO-specific activity were calculated relative to baseline samples.

### Results

32 healthy human participants (100% male, 63% Black, 19% Asian, 13% Caucasian) were enrolled and received a single oral dose of Compound 2 (1, 3, 6, and 10 mg, n=6 each) or placebo (n=2). Compound 2 plasma PK exposure increased in a greater than dose proportional manner between the 3 and 10 mg dose levels. The mean half-life of Compound 2 ranged from 1-3 hours. At 4 hours post-dose, near complete inhibition of plasma SSAO activity was seen in all dose cohorts and continued suppression was detected for up to 1 week after a single dose of Compound 2. Maximum plasma MMA levels increased with Compound 2. No clinically relevant adverse events or laboratory abnormalities were reported.

As shown in Table 8, doses 1, 3, 6, and 10 mg of Compound 2 were all well tolerated.

TABLE 8
Treatment Associated Adverse Events

| | 1 mg of a | 3 mg of a | 10 mg of a | | |
|---|---|---|---|---|---|
| | tosylate salt of | tosylate salt of | 6 mg of a | tosylate salt of | |
| | Compound 2 | Compound 2 | tosylate salt of | Compound 2 | |
| | or placebo | or placebo | Compound 2 | or placebo | All |
| | (n = 8) | (n = 8) | (n = 8) | (n = 8) | (n = 32) |
| Subject | 0 | 0 | 2 (25) | 3 (37.5) | 5 (15.6) |
| incidence of any | | | | | |
| TEAE | | | | | |
| Subject | 0 | 0 | 0 | 1 (12.5) | 1 (3.1) |
| incidence of | | | | | |
| TEAEs | | | | | |
| considered | | | | | |
| possibly | | | | | |
| treatment-related | | | | | |
| TEAE diagnosis | | | | | |
| and frequency | | | | | |
| constipation | 0 | 0 | 0 | 1 (12.5) | 1 (3.1) |
| contact | 0 | 0 | 2 (25) | 0 | 2 (6.3) |
| dermatitis | | | | | |
| dysgeusia | 0 | 0 | 0 | 1 (12.5) | 1 (3.1) |
| headache | 0 | 0 | 0 | 1 (12.5) | 1 (3.1) |
| oral herpes | 0 | 0 | 0 | 1 (12.5) | 1 (3.1) |
| sore throat | 0 | 0 | 1 (12.5) | 0 | 1 (3.1) |
| upper | 0 | 0 | 0 | 1 (12.5) | 1 (3.1) |
| respiratory tract | | | | | |
| infection | | | | | |

Single doses of the tosylate salt of Compound 2 were rapidly cleared from plasma and exhibited greater than dose proportional plasma PK between 3 and 10 mg.

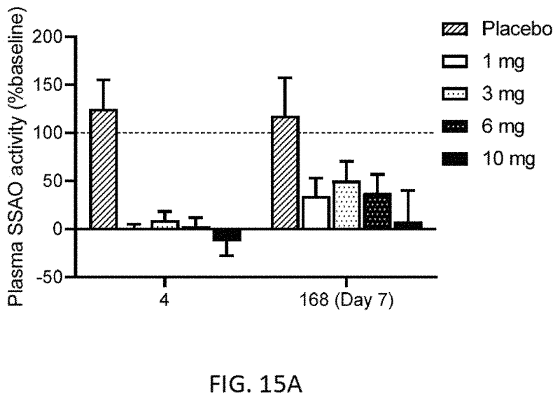

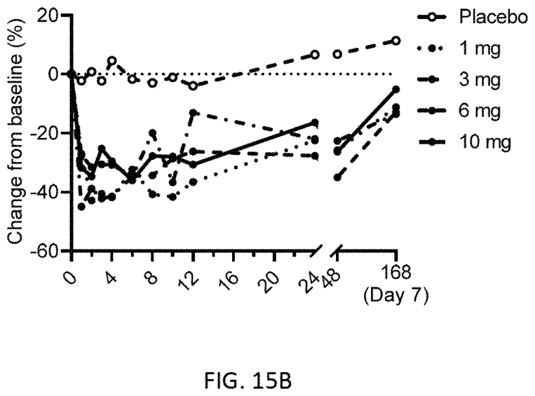

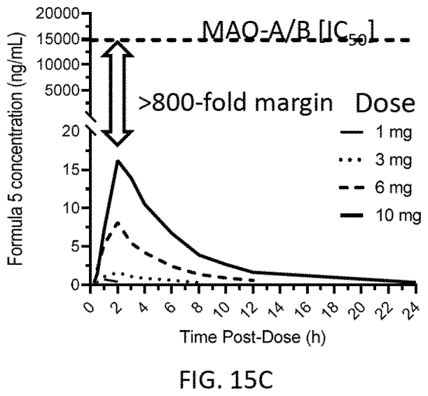

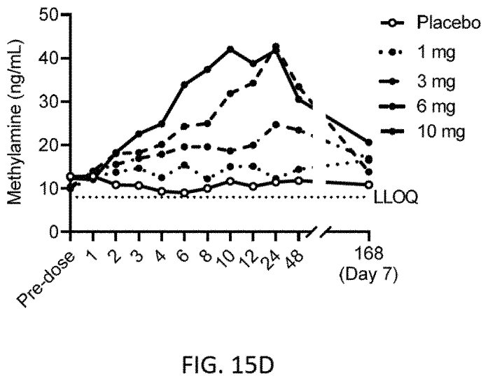

Single doses of Compound 2 rapidly and potently decreased plasma amine oxidase activity in all subjects as shown in FIG. 15A and FIG. 15B. Near complete inhibition of SSAO-specific activity as observed at 4 hours post dose. FIG. 15A and FIG. 15B. Inhibition of plasma SSAO amine oxidase activity and dose-dependent increases in plasma MMA were sustained up to 1 week after single doses of Compound 2, suggesting potent, covalent target engagement and supporting once daily dosing despite a short plasma half-life. FIG. 15A and FIG. 15B.

The concentrations (Cₘₐₓ) for Compound 2 were more than 800 times lower than the IC₅₀ concentrations for MAO-A and MAO-B at all dose levels. FIG. 15C.

TABLE 9
Biochemical activity (IC₅₀ μM)

| SSAO inhibitor | SSAO | MAO-A | MAO-B |
|---|---|---|---|
| Compound 2 | 0.0065 | >50 | >50 |
| BI 1467335 | 0.005 | >100 | 2.7 |
| (PXS-4728A) | | | |

Dose-dependent increases in methylamine were observed, indicating potent plasma SSAO target engagement across the dose range. FIG. 15D.

### Conclusions

Compound 2 was safe and well tolerated in healthy subjects administered a single oral dose ranging from 1 mg to 10 mg. Compound 2 inhibited SSAO activity for up to seven days after a single dose. This suggests that Compound 2 may be effective for treating liver diseases or disorders by selectively inhibiting SSAO. It may also exhibit SSAO activity for seven days after only a single dose, suggesting that daily administration for one week may exert a therapeutic effect for a two-week period.

### Example 11

Animal handling: After arrival, the rats were left for a 2-week acclimation period, during which they were accustomed to the animal facility staff and trained on the procedure of oral gavage. After 2 weeks the animals were put on CHDFD (choline deficient high fat diet) and pre-fed for 4 weeks. Then the rats were started on treatment with test compounds, and 3× per week i.p. NaNO₂ injections, while they remained on CDHFD, for an additional 8 weeks. NaNO₂ was administered at 25 mg/kg i.p. dissolved in PBS 3 times a week (on Mondays, Wednesdays, and Fridays) for 8 weeks while on CDHFD.

Final Sacrifice: Half of the animals of each treatment group were terminated on day 84. The other half of the animals in each group were terminated on the following day, day 85. On the day of sacrifice the animals were fasted for 2 h and received a final treatment with the respective test substance. After the final compound treatment, the animals had no more access to food until sacrifice. At 4 h after the last administration all animals were sacrificed and livers were sampled for further analysis.

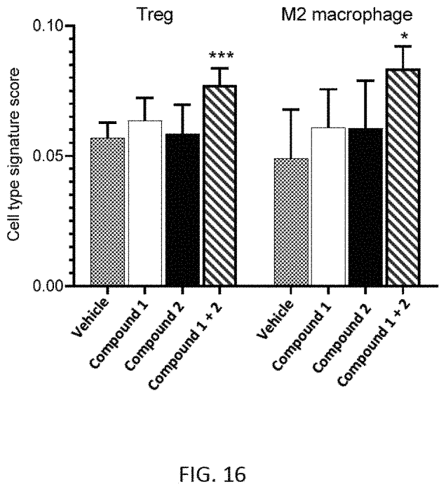

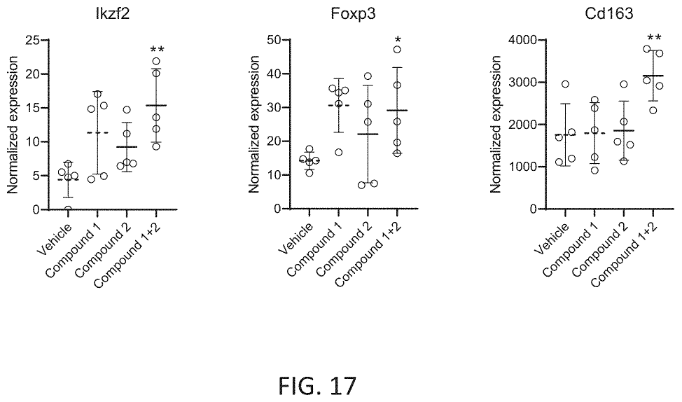

RESULTS: The choline-deficient, high-fat diet (CDHFD) is commonly used to induce a NASH-like phenotype in rodent species. In addition, induction of liver fibrosis by intraperitoneal (IP) injections of sodium nitrite (NaNO₂) in CDHFD rats can be used to model advanced NASH disease. Therefore, the rat CDHFD+NaNO₂ NASH model was used to test the efficacy of Compound 1 alone and in combination with Compound 2. In this model male Wistar rats were fed a CDHFD for 4 weeks to induce disease prior to daily oral drug and triweekly IP NaNO₂ treatment. Following 8 weeks of Compound 1 (3 mg/kg) and Compound 2 (25 mg/kg) dosing as single agents or in combination, liver tissue was processed for whole transcriptome analysis by RNAseq to look for changes in gene expression associated with disease resolution. In NASH, resolution is a complex process that involves liver infiltration of specialized cells of the immune system including regulatory T cells (Treg) and M2 macrophages. Treg and M2 macrophages are involved in immune suppression and reducing inflammation and appear to have a beneficial role in animal models of liver injury including NASH. To look for the presence of these cells, we utilized RNAseq expression data to perform single-sample gene set enrichment analysis (ssGSEA) using cell type specific gene expression signatures to quantitate relative levels of Treg and M2 macrophage infiltration into the liver (FIG. 16). The combination of Compound 1 (3 mg/kg) and Compound 2 (25 mg/kg) showed significantly higher scores for both Treg and M2 macrophages relative to vehicle-treated NASH control animals. In contrast, single agent treated animals were not significantly different from control. These results were verified (FIG. 17) by analysis of individual markers of Treg and M2 macrophages including Foxp3 (Treg), Ikzf2 (Treg), and Cd163 (M2 macrophage). Only the combination of Compound 1 (3 mg/kg) and Compound 2 (25 mg/kg) showed significantly higher expression of markers associated with Treg and M2 macrophage cells. Taken together these data suggest that the combination of Compound 1, an FXR agonist, and Compound 2, an inhibitor of SSAO, resulted in increased expression of immune cell markers in the liver that are associated with NASH resolution. Given their distinct mechanisms of action, Compound 1 and Compound 2 could provide complementary benefits when used in combination to accelerate NASH resolution processes.

These results demonstrate that the combination of a FXR agonist and an SSAO inhibitor combine to have an effect that is greater than either of the two drugs administered singly.

### Example 12

3 groups of 8 healthy participants were randomized to receive multiple once daily (QD) doses of Compound 2 or matching placebo in a 3:1 ratio for 7 days (1 mg and 4 mg) or 14 days (10 mg). Plasma levels of Compound 2 and PD biomarkers (plasma amine oxidase activity and methylamine levels) were determined at pre-dose and various timepoints post-dose. Safety was assessed for up to 14 days after last dose.

No clinically relevant adverse events or laboratory abnormalities were reported. Compound 2 plasma PK exposure increases were greater than dose proportional between dose groups on Day 1, and significant accumulation at each dose level was observed after multiple QD doses. The accumulation ratio between the first and last day of dosing decreased as dose increased. Steady state was achieved in the highest dose cohort (10 mg) after 7 days. Compound 2 half-life increased with dose, consistent with a saturable target-mediated clearance. Near complete inhibition of plasma SSAO activity was seen on Day 1 in all dose cohorts and continued suppression was detected for up to 2 weeks after last dose in the 10 mg cohort. Plasma methylamine levels increased in a greater than dose proportional manner.

Compound 2 was safe and well tolerated in healthy subjects when administered up to 10 mg QD for 14 days. Steady state levels of Compound 2 were achieved after 7 days of dosing supporting a QD dosing regimen. Near complete inhibition of plasma SSAO amine oxidase activity and dose-dependent increases in plasma MMA were sustained up to 2 weeks after cessation of dosing, suggesting that daily administration of Compound 2 for two weeks may exert a therapeutic effect for a two-week period after cessation of dosing.

### Example 13

A study was performed to show the beneficial effects of combining a FXR agonist and a SSAO inhibitor in a rat model of NASH.

Animal handling: After arrival, the rats were left for a 2-week acclimation period, during which they were accustomed to the animal facility staff and trained on the procedure of oral gavage. After 2 weeks the animals were placed on a choline deficient high fat diet (CDHFD) and pre-fed for 4 weeks to induce steatosis and a NASH-like disease phenotype. Rats were then treated with test compounds for an additional 8 weeks while on CDHFD. Concomitant with compound treatment, rats were administered sodium nitrite (NaNO₂, 25 mg/kg dissolved in PBS) by triweekly intraperitoneal (IP) injection to induce liver fibrosis.

Final Sacrifice: Half of the animals of each treatment group were terminated on day 84. The other half of the animals in each group were terminated on the following day, day 85. On the day of sacrifice the animals were fasted for 2 hours and received a final treatment with the respective test substance. After the final compound treatment, the animals had no more access to food until sacrifice. At 4 hours after the last administration all animals were sacrificed and livers were sampled for further analysis.

Sampling and analysis: Small liver pieces were harvested into RNAlater (Thermo Fisher Scientific Dreieich Germany) and stored at −20° C. prior to RNA sequencing (RNAseq) at MedGenome Inc. RNAseq analysis was performed on liver tissue by Illumina sequencing using standard methodologies. Briefly, RNAseq libraries (n=5 per group) were generated using Illumina Truseq stranded mRNA kits and sequencing was performed on a NovaSeq 6000 sequencer. Alignment was performed using STAR (v2.7.3a) aligner and reads mapping to ribosomal and mitochondrial genome were removed prior to alignment. Raw read counts were estimated using HTSeq (v0.11.1) and normalized using DESeq2 (v2.22.2). Differentially expressed genes (DEGs) were determined using DESeq2 (R Bioconductor package).

RESULTS: The choline-deficient, high-fat diet (CDHFD) is commonly used to induce a NASH-like phenotype in rodent species. In addition, induction of liver fibrosis by intraperitoneal (IP) injections of sodium nitrite (NaNO₂) in CDHFD rats can be used to model advanced NASH disease. Therefore, the rat CDHFD+NaNO₂ NASH model was used to test the efficacy of Compound 1 alone and in combination with Compound 2. In this model, male Wistar rats were fed a CDHFD for 4 weeks to induce disease prior to daily oral drug and triweekly IP NaNO₂ treatment. Following 8 weeks of Compound 1 (3 mg/kg) and Compound 2 (25 mg/kg) dosing as single agents or in combination, liver tissue was processed for whole transcriptome analysis by RNAseq. Table 10 shows the total number and change direction (i.e., up or down relative to vehicle control) of differentially expressed genes (DEGs) identified in CDHFD+NaNO₂ rats treated with Compound 1 (3 mg/mg), Compound 2 (25 mg/kg), or the combination of Compound 1 (3 mg/kg) and Compound 2 (25 mg/kg). Using an absolute fold-change cutoff of ≥1.5-fold and adjusted p-value of <0.01, 309 DEGs were identified in Compound 1 treatment group, 847 DEGs were identified in Compound 2 treated animals, and 1351 DEGs were identified in the combination treatment group. These results suggest that the combination treatment resulted in at least additive effects on the total number of DEGs relative to single agent treatment groups.

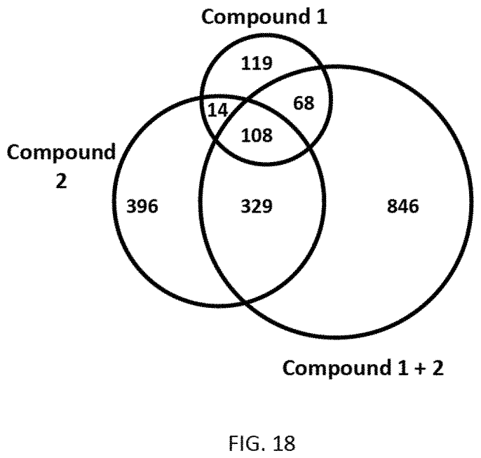

Surprisingly, a larger number of upregulated DEGs were observed in the combination treatment group relative to individual treatment arms. FIG. 18 shows the number and overlap of DEGs (vs. vehicle NASH control) identified in each treatment group using absolute fold-change and adjusted p-value cutoffs of ≥1.5 and <0.01, respectively.

TABLE 10
Differentially expressed genes (DEGs)

| Treatment group | Down DEGs | Up DEGs | Total DEGs |
|---|---|---|---|
| Compound 1 (3 mg/kg) | 118 | 191 | 309 |
| Compound 2 (25 mg/kg) | 641 | 206 | 847 |
| Compound 1 (3 mg/kg) + | 724 | 627 | 1351 |
| Compound 2 (25 mg/kg) | | | |

Number of DEGs identified (vehicle NASH control vs. treatment) identified for each treatment group. Adjusted p-value <0.01 and fold-change ≥1.5-fold.

We next examined the differential expression of genes associated with lipid metabolism and triglyceride accumulation that were previously described (Shepherd E, Karim S, Newsome P, and Lalor P., Inhibition of vascular adhesion protein-1 modifies hepatic steatosis in vitro and in vivo. World J Hepatol. 2020 12(11): 931-948). Compound 2 treatment resulted in statistically significant changes in the expression of genes related to lipid metabolism and fatty-acid transportation including V1d1r, Fabp2, Vegfc, Ld1rap1, Ld1r, Ppargc1a, and S1c27a5 (Table 11, denoted by asterisk). Of these, V1d1r, Fabp2, and S1c27a5 were changed by ≥1.5-fold (shown in bold). Only Fabp2 was significantly differentially expressed upon treatment with Compound 1. Interestingly, the combination of Compound 1 and Compound 2 resulted in substantially more DEGs related to lipid metabolism and fatty-acid transportation than either single agent treatment group. Moreover, several genes were differentially expressed by ≥1.5-fold relative to vehicle control, including V1d1r, Fabp2, I11r2, Ppara, Ld1r, Ppargc1a, Rxra, and S1c27a5.

Taken together these data suggest that the combination of Compound 1, an FXR agonist, and Compound 2, an inhibitor of SSAO, resulted in significant changes in the expression of genes involved in lipid metabolism and fatty-acid transport. Moreover, the pattern of gene expression changes is largely consistent with an enhanced anti-steatotic effect relative to treatment with Compound 1 alone. Given their distinct mechanisms of action, Compound 1 and Compound 2 could provide complementary benefits when used in combination to accelerate NASH resolution processes.

TABLE 11
Differentially expressed genes associated with lipid
metabolism and fatty acid transport

| | Differential gene expression analysis (log2-fold | | |
|---|---|---|---|
| | change) relative to vehicle control | | |
| | Compound | Compound | Compound 2 + |
| | 2 | 1 | Compound 1 |
| Gene | (25 mg/kg) | (3 mg/kg) | (25 mg/kg + 3 mg/kg) |
| Vldlr | −1.6* | −0.58 | −1.17* |
| Fabp2 | −1.02* | −1.02* | −1.2* |
| I11r2 | −0.45 | −0.05 | −0.95* |
| Vegfc | −0.45* | −0.28 | −0.54* |
| Lrp2 | 0.08 | 0.33 | 0.32* |
| Irs2 | 0.13 | 0.27 | 0.41* |
| Vegfa | 0.23 | 0.48 | 0.41* |
| Lrp1 | 0.25 | 0.51 | 0.48* |
| Irs1 | 0.26 | −0.03 | 0.45* |
| Ppara | 0.32 | 0.36 | 0.68* |
| Slc27a1 | 0.33 | 0.16 | 0.51* |
| Ldlrap1 | 0.38* | −0.07 | 0.32* |
| Ldlr | 0.41* | 0.4 | 0.67* |
| Ppargc1a | 0.51* | 0.25 | 0.85* |
| Rxra | 0.51 | 0.03 | 0.62* |
| Slc27a5 | 0.68* | 0.5 | 0.81* |

Gene expression analysis (RNAseq) in the liver of CDHFD+NaNO₂ rats. Log₂-fold-change relative to vehicle control for genes involved in lipid metabolism and fatty-acid transportation. Negative change direction (−) indicates decreased expression by treatment relative to vehicle; positive change direction indicates increased gene expression relative to vehicle control. Absolute fold-change values ≥1.5-fold (i.e., log₂-fold change ≥0.6 or ≤−0.6) indicated in bold. *p-value <0.05.

### Example 14

A randomized, double-blind, placebo-controlled study is conducted to evaluate the safety and efficacy of combination treatments, for example, Compound 1 and Compound 2. Subjects with NASH are treated once daily with the FXR agonist and the SSAO inhibitor in combination for 12 or 48 weeks. Liver fat is monitored by MRI-PDFF, and serum-based non-invasive fibrosis or NASH markers such as Pro-C3, TIMP-1, PIIINP, CK-18, and ALT, are measured. Side effects such as pruritus and LDL-C cholesterol levels are also monitored.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing invention has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the invention.

1. A method of treating non-alcoholic steatohepatitis (NASH) in a patient in need thereof, comprising administering to the patient a Farnesoid X Receptor (FXR) agonist and a Semicarbazide-Sensitive Amine Oxidase (SSAO) inhibitor, wherein the FXR agonist is a compound of formula (1): 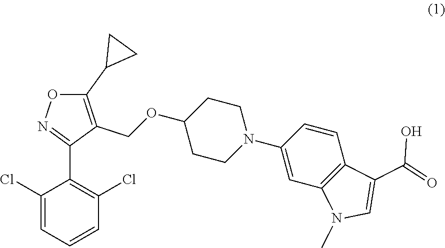 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and the SSAO inhibitor is a compound of formula (2): 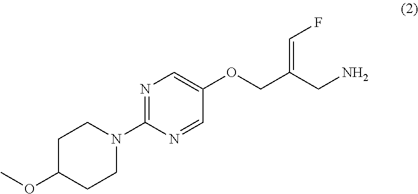 [SMILES: COC1CCN(c2ncc(OC/C(=C/F)CN)cn2)CC1]
or a pharmaceutically acceptable salt thereof.

2-14. (canceled)

15. The method of claim 1, wherein the FXR agonist and the SSAO inhibitor are administered simultaneously.

16. The method of claim 1, wherein the FXR agonist and the SSAO inhibitor are administered sequentially.

17. The method of claim 1, wherein the administration does not result in pruritus in the patient at a severity of Grade 2 or more.

18. The method of claim 1, wherein the administration does not result in pruritus in the patient at a severity of Grade 1 or more.

19. The method of claim 1, wherein the patient also has a cardiovascular disorder.

20. The method of claim 1, wherein the patient also has diabetes mellitus.

21.-22. (canceled)

23. The method of claim 1, wherein the FXR agonist is administered orally once daily or twice daily.

24. The method of claim 23, wherein the SSAO inhibitor is administered orally once daily or twice daily.

25-40. (canceled)

41. A method of reducing hepatic steatosis in a patient in need thereof, comprising administering to the patient a therapeutically effective amount of a FXR agonist and a therapeutically effective amount of a SSAO inhibitor, wherein the FXR agonist is a compound of formula (1): 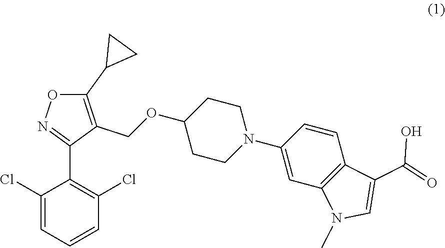 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and the SSAO inhibitor is a compound of formula (2): 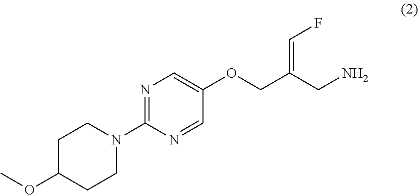 [SMILES: COC1CCN(c2ncc(OC/C(=C/F)CN)cn2)CC1]
or a pharmaceutically acceptable salt thereof.

42. A method of treating a disease or condition characterized by steatosis of the liver, said method comprising administering to a patient in need of treatment a therapeutically effective amount of a FXR agonist and a therapeutically effective amount of a SSAO inhibitor, wherein the FXR agonist is a compound of formula (1): 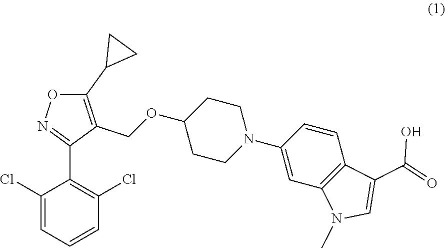 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and the SSAO inhibitor is a compound of formula (2): 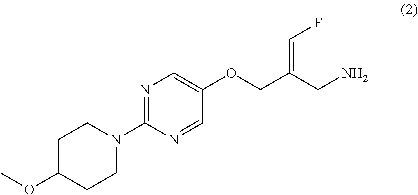 [SMILES: COC1CCN(c2ncc(OC/C(=C/F)CN)cn2)CC1]
or a pharmaceutically acceptable salt thereof.

43-47. (canceled)

48. The method of claim 1, wherein the patient has liver fibrosis.

49-70. (canceled)

71. The method of claim 1, wherein the compound of formula (1), or a pharmaceutically salt thereof, is administered to the patient at a dose from about 1 mg to 15 mg daily and the compound of formula (2), or a pharmaceutically salt thereof, is administered to the patient at a dose from about 1 mg to about 20 mg daily.

72. The method of claim 71, wherein the compound of formula (1), or a pharmaceutically salt thereof, and the compound of formula (2), or a pharmaceutically salt thereof, are each administered once daily to the patient.

73. The method of claim 1, wherein the compound of formula (1), or a pharmaceutically salt thereof, is administered to the patient at a dose from about 5 mg to 15 mg daily and the compound of formula (2), or a pharmaceutically salt thereof, is administered to the patient at a dose from about 1 mg to about 5 mg daily.

74. The method of claim 73, wherein the compound of formula (1), or a pharmaceutically salt thereof, and the compound of formula (2), or a pharmaceutically salt thereof, are each administered once daily to the patient.

75. The method of claim 1, wherein the compound of formula (1), or a pharmaceutically salt thereof, is administered to the patient at a dose from about 5 mg to 15 mg daily and the compound of formula (2), or a pharmaceutically salt thereof, is administered to the patient at a dose from about 4 mg to about 10 mg daily.

76. The method of claim 75, wherein the compound of formula (1), or a pharmaceutically salt thereof, and the compound of formula (2), or a pharmaceutically salt thereof, are each administered once daily to the patient.

77. The method of claim 1, wherein the compound of formula (1), or a pharmaceutically salt thereof, is administered to the patient at a dose from about 5 mg to 15 mg daily and the compound of formula (2), or a pharmaceutically salt thereof, is administered to the patient at a dose from about 10 mg to about 20 mg daily.

78. The method of claim 77, wherein the compound of formula (1), or a pharmaceutically salt thereof, and the compound of formula (2), or a pharmaceutically salt thereof, are each administered once daily to the patient.

79. The method of claim 1, wherein the FXR agonist is a compound of formula (1):

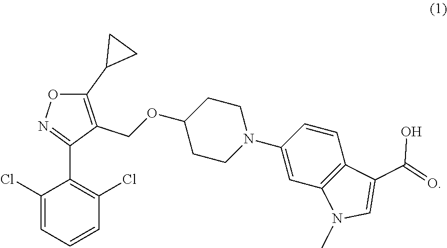 [SMILES: Cn1cc(C(=O)O)c2ccc(N3CCC(OCc4c(-c5c(Cl)cccc5Cl)noc4C4CC4)CC3)cc21]

80. The method of claim 1, wherein the SSAO inhibitor is a 4-methlylbenzenesulfonate salt of the compound of formula (2).

81. The method of claim 79, wherein the SSAO inhibitor is a 4-methlylbenzenesulfonate salt of the compound of formula (2).

82. The method of claim 81, wherein the FXR agonist is administered to the patient at a dose from about 5 mg to 15 mg daily and the SSAO inhibitor is administered to the patient at a dose from about 4 mg to about 10 mg daily.

83. A fixed-dose pharmaceutical composition for oral administration, comprising an FXR agonist of the compound of formula (1): 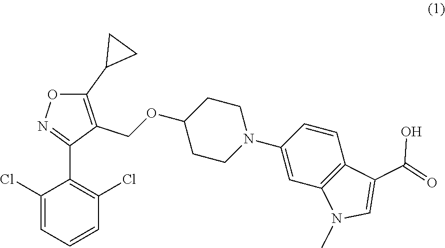 [SMILES: Cn1cc(C(=O)O)c2ccc(N3CCC(OCc4c(-c5c(Cl)cccc5Cl)noc4C4CC4)CC3)cc21]
or a pharmaceutically acceptable salt thereof, and an SSAO inhibitor of the compound of formula (2): 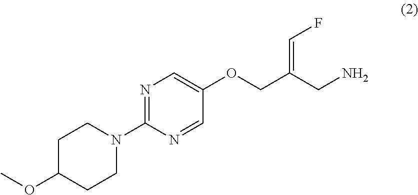 [SMILES: COC1CCN(c2ncc(OC/C(=C/F)CN)cn2)CC1]
or a pharmaceutically acceptable salt thereof.

84. The fixed-dose pharmaceutical composition of claim 83, wherein the composition comprises from about 1 mg to about 15 mg of the compound of formula (1), or a pharmaceutically salt thereof, and from about 1 mg to about 20 mg of the compound of formula (2), or a pharmaceutically salt thereof.

85. The fixed-dose pharmaceutical composition of claim 84, wherein the FXR agonist is a compound of formula (1): 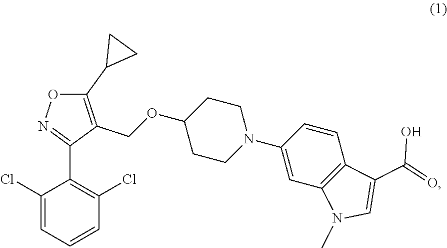 [SMILES: Cn1cc(C(=O)O)c2ccc(N3CCC(OCc4c(-c5c(Cl)cccc5Cl)noc4C4CC4)CC3)cc21]
and the SSAO inhibitor is a 4-methlylbenzenesulfonate salt of the compound of formula (2).

86. The fixed-dose pharmaceutical composition of claim 83, wherein composition comprises from about 5 mg to about 15 mg of the compound of formula (1), or a pharmaceutically salt thereof, and from about 4 mg to about 10 mg of the compound of formula (2), or a pharmaceutically salt thereof.

87. The fixed-dose pharmaceutical composition of claim 86, wherein the FXR agonist is a compound of formula (1): 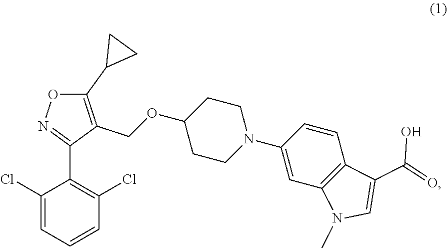 [SMILES: Cn1cc(C(=O)O)c2ccc(N3CCC(OCc4c(-c5c(Cl)cccc5Cl)noc4C4CC4)CC3)cc21]
and the SSAO inhibitor is a 4-methlylbenzenesulfonate salt of the compound of formula (2).
